(19) United States
(12) Patent Application Publication
(10) Pub. No.: US 2023/0277068 A1
(43) Pub. Date: Sep. 7, 2023

(54) SYSTEMS AND METHODS FOR CLASSIFYING CRITICAL HEART DEFECTS

(71) Applicant: THE REGENTS OF THE UNIVERSITY OF CALIFORNIA, Oaklarnd, CA (US)

(72) Inventors: Heather Siefkes, Davis, CA (US); Satyanarayana Lakshminrusimha, Davis, CA (US); Chen-Nee Chuah, Davis, CA (US); Zhengfeng Lai, Davis, CA (US)

(21) Appl. No.: 17/919,159

(22) PCT Filed: Apr. 16, 2021
(86) PCT No.: PCT/US2021/027837

§ 371 Date: Oct. 14, 2022

Related U.S. Application Data
(60) Provisional application No. 63/011,998, filed on Apr. 17, 2020.

(51) Int. Cl. A61B 5/0205; A61B 5/1455; A61B 5/00; A61B 5/026; G16H 50/20; G06N 20/20; G06N 20/10

(52) U.S. Cl. CPC A61B 5/0205; A61B 5/026; A61B 5/14551; A61B 5/681; A61B 5/7267; A61B 5/7275; G06N 20/10; G06N 20/20; G16H 50/20; A61B 5/02416; A61B 5/6829; A61B 2503/045

(57) ABSTRACT

Disclosed are systems and methods for classifying heart defects using a predictive model based on a set of parameters that includes oxygen saturation (Sp02), perfusion index (PIx), heart rate (HR) data, radiofemoral delay, and/or photoplethysmography (PPG) slope.

### CROSS-REFERENCE TO RELATED APPLICATIONS

This application claims priority under 35 U.S.C. § 119(e) to U.S. Provisional Application No. 63/011998, filed Apr. 17, 2020, which is incorporated herein by reference in its entirety.

### STATEMENT REGARDING FEDERALLY SPONSORED RESEARCH

This invention was made with Government support under Grant No. 1R21HD099239 and UL1 TR001860 with linked award KL2 TR001859 awarded by the National Institutes of Health (NIH). The Government has certain rights in the invention.

### BACKGROUND

Congenital heart disease is the most common birth defect affecting approximately 0.8% of all births. Critical congenital heart disease (CCHD) accounts for approximately 20% of congenital heart disease and is life threatening if not timely diagnosed. In fact, prior to universal oxygen-saturation based CCHD screening, 25% of CCHD were diagnosed after hospital discharge, with some diagnosed at autopsy. Oxygen-saturation screening has since reduced mortality associated with CCHD and helped with earlier diagnosis, but nearly 900 neonates with CCHD remain undiagnosed annually in the United States (US). Coarctation of the aorta (CoA) is the most commonly missed CCHD defect despite oxygen saturation screening as it is associated with poor systemic perfusion without hypoxemia. Late diagnosis of defects such as CoA can be particularly detrimental. Preliminary analysis of CCHD defects missed by oxygen-saturation screening demonstrates that 18% of newborns with late CoA diagnosis die, some before surgery can be done. More than 50% of deaths due to missed CCHD occur before corrective surgery, either at home or shortly after arriving to the Emergency Department.

### SUMMARY

The following summary is illustrative only and is not intended to be in any way limiting. In addition to the illustrative aspects, embodiments, and features described above, further aspects, embodiments, and features will become apparent by reference to the below drawings and the detailed description.

In one aspect, various embodiments may relate to a computer-implemented method comprising: obtaining, via a first oximeter probe secured to an upper extremity (such as a hand or wrist, preferably a right hand or right wrist, an upper arm, a lower arm, or to a suitable preductal site in newborns or other infants) of a patient and/or a second oximeter probe secured to a lower extremity (such as a foot or ankle, an upper leg, a lower leg, or to a suitable postductal site in infants) of a patient, a plurality of physiological measurements from the patient; applying a predictive model to the plurality of physiological measurements from the patient to generate a classification corresponding to a vascular condition, the predictive model having been trained, using a machine learning system, by: acquiring, using one or more pulse oximeters, physiological readings from subjects in a study cohort; extracting a set of features from the physiological readings to generate a training dataset based on the physiological readings from the subjects in the study cohort; and applying machine learning techniques to the training dataset to train the predictive model such that the predictive model is configured to accept the plurality of physiological measurements and generate a classification corresponding to the vascular condition, wherein applying the machine learning techniques comprises performing automated feature selection to identify a subset of the set of features and refitting the predictive model based on the subset of features, wherein the subset of features corresponds to the plurality of physiological measurements; and outputting or storing the classification in association with the patient. In various embodiments, the first oximeter probe (secured, e.g., to an upper extremity or preductally, such as a hand or wrist) or the second oximeter probe (secured, e.g., to a lower extremity or postductally, such as a foot or ankle), but not both the first and second oximeter probes, may be used to obtain physiological measurements from the patient. In certain embodiments, the first oximeter probe (secured to the upper extremity or preductally) may be excluded, and only the second oximeter probe (secured to the lower extremity or postductally) may be used to obtain physiological measurements from the patient. In certain embodiments, one or more additional oximeter probes (secured to upper and/or lower extremities, and/or preductally and/or postductally) may be used to obtain physiological measurements from the patient. As used herein, “hand” refers to any part of the hand, including the palm and/or any individual finger or combination of fingers, “foot” includes any part of the foot, including the sole and/or any individual toe or combination of toes. As used herein, “wrist” refers to the anatomical region surrounding the carpus including distal parts of forearm bones and proximal parts of the metacarpus and wrist joints. As used herein, “upper extremity” or “arm” includes the upper arm (the region of the arm between the shoulder and the elbow), the lower arm or forearm (the region between the elbow and the wrist), the wrist, and/or the hand. As used herein, “lower extremity” or “leg” includes the upper leg (the region between the hip and the knee), the lower leg (the region between the knee and the ankle), the ankle, and the foot.

In example embodiments, a computer-implemented method comprises: (A) obtaining, via a first oximeter probe secured to an upper extremity of a patient and/or a second oximeter probe secured to a lower extremity of the patient, a plurality of physiological measurements from the patient; (B) applying a predictive model to the plurality of physiological measurements from the patient to generate a classification corresponding to a vascular condition, the predictive model having been trained, using a machine learning system, by: (i) acquiring, using one or more pulse oximeters, physiological readings from subjects in a study cohort; (ii) extracting a set of features from the physiological readings to generate a training dataset based on the physiological readings from the subjects in the study cohort; and (iii) applying machine learning techniques to the training dataset to train the predictive model such that the predictive model is configured to accept the plurality of physiological measurements and generate a classification corresponding to the vascular condition, wherein applying the machine learning techniques comprises performing automated feature selection to identify a subset of the set of features and refitting the predictive model based on the subset of features, wherein the subset of features corresponds to the plurality of physiological measurements; and (C) outputting or storing the classification in association with the patient.

Regarding generation of the classification generated, and steps involved in application of the model to the measurements, in various embodiments supervised machine learning may be employed with a dataset that incorporates the final classification of the enrolled subjects in the cohort. The training dataset provided to the model may include labels to indicate, for example, which data corresponds to newborns with or without a vascular condition and which type of vascular condition (e.g., CHD or CCHD). In certain embodiments, 80/20 splits of the data may be used, with iterations in which 80% of the data is used for training the data and 20% of the data is used for testing the model.

Regarding training dataset generation and feature extraction, various embodiments record the pulse oximetry raw data. This includes numerical values for HR, SpO2, and PAI per the device sampling frequency and then also the data to recreate the photoplethysmography (PPG) waveform. Various embodiments then identify HR, PAI, and SpO2 values that likely correspond to artifact and not physiologic-represented values. After removing the values likely associated with artifact, various embodiments extract a set of features (e.g., the minimum, maximum, median, mean, and variance values for HR, SpO2, and PAI) from the physiological readings for each patient. Various embodiments then repeat this process for each subject, such that each subject will have a corresponded set of features. Various embodiments then combine all features from each subject to form a training dataset that also uses the corresponding final classification indicating, for example, “with” or “without” the vascular condition (e.g., CHD or CCHD).

Regarding the machine learning fitting, potential embodiments may employ a loss function that calculates the distance between the model’s output and the ground truth (labels, healthy or CCHD). The training process may be used to minimize the loss to make the model’s output as close as possible to the ground truth. Additionally, as discussed herein, various embodiments create and test the algorithm using 80/20 splits to randomly divide the data for training and testing and then 5-fold validation.

Regarding automated feature selection, various embodiments begin by using all features in the feature set (e.g., the minimum, maximum, median, mean, and variance values for HR, SpO2, and PAI) as the input to fit the predictive model, recording the results in this setting. Various embodiments then may recursively remove features to form a subset of the original feature set. Various embodiments may then fit the subset into the predictive model and record the results in this setting. This step may be repeated until all subsets of the original features have been fed into the predictive model, after which various embodiments may pick up the subset of features that have the best results recorded.

In various embodiments, the vascular condition may be a congenital heart disease. In various embodiments, the patient and the subjects in the cohort may be newborns and/or infants. In various embodiments, the machine learning techniques may comprise a random forest classifier. In various embodiments, the machine learning techniques may comprise logistic regression. In various embodiments, the machine learning techniques may comprise a Naive Bayes Classifier. In various embodiments, the machine learning techniques may comprise a K-Nearest Neighbours (k-NN) algorithm. In various embodiments, the machine learning techniques may comprise a Decision Tree. In various embodiments, the machine learning techniques may comprise a Support Vector Machine algorithm. In various embodiments, the machine learning techniques may comprise a Gradient Boosting Classifier. In various embodiments, the machine learning techniques may comprise an ensemble of a random forest classifier and logistic regression. In various embodiments, the machine learning techniques may comprise an ensemble, for example, of a random forest classifier, logistic regression, a Naive Bayes Classifier, a K-Nearest Neighbours algorithm, a Decision Tree, a Support Vector Machine algorithm, and/or a Gradient Boosting Classifier. In various embodiments, the method may further comprise securing the first oximeter probe to an upper extremity, preferably the right hand of the patient, and/or securing the second oximeter probe to a lower extremity, preferably either foot of the patient. In various embodiments, the method may further comprise securing only the second oximeter probe to a lower extremity, such as a foot of the patient, but not the first oximeter probe. In various embodiments, the subset of features may comprise oxygen saturation (SpO2), heart rate, and/or perfusion amplitude index (PAI) (e.g.: SpO2 and HR; SpO2 and PIx; HR and PIx; or SpO2, HR, and PIx). In various embodiments, performing automated feature selection may comprise performing Recursive Feature Elimination (RFE). In various embodiments, RFE may be performed with sensitivity as the score to be optimized. In various embodiments disclosed herein, the physiological readings from the subjects may be acquired over a predetermined time period. In various embodiments, the time period may be at least about one minute, at least about two minutes, at least about three minutes, at least about four minutes, or at least about five minutes, etc. In certain embodiments, the time periods for patients and/or subjects may be any time period between about one minute to about ten minutes and any ranges therein, such as about three minutes to about seven minutes. In various embodiments, the method may further comprise displaying, on a display screen, physiological readings sensed via the first and second oximeter probes in real time or near real time.

In another aspect, various potential embodiments may relate to a method comprising using a machine learning system to train a machine learning predictive model by: acquiring, using one or more pulse oximeters, physiological readings from subjects in a study cohort for a time period; extracting a set of features from the physiological readings to generate a training dataset based on the physiological readings from the subjects in the study cohort; and applying machine learning techniques to the training dataset to train the predictive model such that the predictive model is configured to accept data based on a plurality of physiological measurements from patients and generate classifications corresponding to a vascular condition, wherein the training dataset comprises a set of features, and wherein applying the machine learning techniques comprises performing automated feature selection to identify a subset of the set of features and refitting the predictive model based on the subset of features.

In various embodiments, the vascular condition may be a congenital heart disease. In various embodiments, the subjects in the cohort may be newborns and/or infants. In various embodiments, a first oximeter probe may be secured to an upper extremity, preferably the right hand or wrist of each of the subjects, or a preductal site, and/or a second oximeter probe may be secured to a lower extremity, preferably either foot or ankle, or a postductal site, of each of the subjects. In various embodiments, the subset of features comprises oxygen saturation (SpO2), heart rate (HR), and/or perfusion amplitude index (PAI) (e.g.: SpO2 and HR; SpO2 and PIx; HR and PIx; or SpO2, HR, and PIx). As used herein, PAI is synonymous with perfusion index (PIx), as one is a factor of 100 of the other, and thus, the two may be used interchangeably. In various embodiments, performing automated feature selection may comprise performing Recursive Feature Elimination (RFE). In various embodiments, the physiological readings from the subjects may be acquired over a time period of, for example, at least about one, two, three, four, five, six, or seven minutes. In any of the embodiments disclosed herein, the method may further comprise acquiring, using one or more pulse oximeters, a plurality of physiological readings from a patient; and applying the predictive model to a plurality of physiological measurements based on the physiological readings from the patient to generate a classification corresponding to the vascular condition.

In another aspect, various potential embodiments may relate to a method comprising: acquiring, by one or more processors, using one or more pulse oximeters, oxygen saturation (SpO2), heart rate (HR), and/or perfusion index (PIx) data (e.g.: SpO2 and HR; SpO2 and PIx; HR and PIx; or SpO2, HR, and PIx) from subjects in a study cohort to generate a training dataset; applying machine learning techniques to the training dataset to train a predictive model such that the predictive model is configured to accept SpO2, HR and/or PIx data and generate a classification corresponding to a vascular condition; acquiring, by the one or more processors, using one or more pulse oximeters, SpO2, HR, and/or PIx data from a patient; and applying, by the one or more processors, the predictive model to the SpO2, HR and/or PIx data from the patient to generate the classification corresponding to the vascular condition.

In various embodiments, the vascular condition is a congenital heart defect, and the subjects and the patient are newborns and/or infants. In various embodiments, the classification may correspond to at least one of a presence of the vascular condition or a severity of the vascular condition.

In another aspect, various embodiments relate to a computer-implemented method to classify congenital heart defects in newborns and/or infants. The method may comprise: acquiring, by one or more processors, using one or more pulse oximeters, oxygen saturation (SpO2), heart rate (HR), and/or perfusion index (PIx) data (e.g.: SpO2 and HR; SpO2 and PIx; HR and PIx; or SpO2, HR, and PIx) from a study cohort to generate a training dataset; using the training dataset to train a predictive model such that the predictive model is configured to accept SpO2, HR, and/or PIx data and generate a classification as to whether a congenital heart defect is detected; acquiring, by the one or more processors, using one or more pulse oximeters, SpO2, HR, and/or PIx data from a subject; applying, by the one or more processors, the predictive model to the SpO2, HR and/or PIx data from the subject to generate the classification as to whether the congenital heart defect is detected in the subject.

In various embodiments, the predictive model may be further configured to accept radiofemoral delay for use in generating the classification. In various embodiments, the radiofemoral delay may be based on simultaneous hand and foot measurements. In various embodiments, the predictive model may be further configured to accept photoplethysmography (PPG) waveform data for use in generating the classification. In various embodiments, the PPG waveform data may comprise PPG waveform slope. In various embodiments, the PPG waveform data may comprise one or more PPG waveform images. In various embodiments, the predictive model may be further configured to accept heart rate data for use in generating the classification. In various embodiments, the heart rate data may comprise heart rate measurements. In various embodiments, the heart rate data may comprise heart rate variability data.

In another aspect, various embodiments relate to a system comprising a computing device and one or more pulse oximeters. The computing device may comprise a controller configured to: acquire, from the one or more pulse oximeters, oxygen saturation (SpO2), heart rate (HR), and/or perfusion index (PIx) measurements (e.g.: SpO2 and HR; SpO2 and PIx; HR and PIx; or SpO2, HR, and PIx) from a patient; and apply a predictive model to a set of patient data comprising the SpO2, HR, and/or PIx measurements from the patient to generate an classification as to whether a heart defect is detected in the patient.

In various embodiments, the controller may be configured to train the predictive model by: acquiring, from one or more pulse oximeters, SpO2, HR, and/or PIx data from a study cohort to generate a training dataset; and using the training dataset to train the predictive model such that the predictive model is configured to accept SpO2, HR, and/or PIx data and generate the classification as to whether the heart defect is detected.

In various embodiments, the controller may be further configured to obtain radiofemoral delay, wherein the set of patient data may further comprise the radiofemoral delay. In various embodiments, radiofemoral delay may be based on simultaneous hand and foot measurements. In various embodiments, the controller may be further configured to obtain photoplethysmography (PPG) waveform data, wherein the set of patient data may further comprise the PPG waveform data. In various embodiments, the PPG waveform data may comprise PPG waveform slope. In various embodiments, the controller may be further configured to obtain heart rate data, wherein the set of patient data may further comprise the heart rate data. In various embodiments, the heart rate data may comprise heart rate measurements, wherein the set of patient data may further comprise the heart rate measurements. In various embodiments, the heart rate data may comprise heart rate variability data, wherein the set of patient data may further comprise the heart rate variability data. In various embodiments, the controller may be further configured to obtain PPG waveform data, wherein the set of patient data may further comprise the PPG waveform data. In various embodiments, the PPG waveform data may comprise a PPG waveform slope, wherein the set of patient data may further comprise the PPG waveform slope. In various embodiments, the PPG waveform data may comprise a PPG waveform image, wherein the set of patient data may further comprises the PPG waveform image.

In another aspect, various embodiments relate to a computer-implemented method. The method may comprise: acquiring, by a controller of a computing device using one or more pulse oximeters, oxygen saturation (SpO2), heart rate (HR), and/or perfusion index (PIx) measurements (e.g.: SpO2 and HR; SpO2 and PIx; HR and PIx; or SpO2, HR, and PIx) from a patient; and applying, by the controller, a predictive model to a set of patient data comprising the SpO2, HR, and PIx measurements from the patient to generate a classification as to whether a heart defect is detected in the patient.

In any of the embodiments disclosed herein, the predictive model may be trained by: acquiring, by the controller, using one or more pulse oximeters, SpO2, HR, and PIx data from a study cohort to generate a training dataset; and using, by the controller, the training dataset to train the predictive model such that the predictive model is configured to accept SpO2, HR, and PIx data and generate the classification as to whether the heart defect is detected.

In various potential embodiments, two independent perfusion and oxygenation monitors (oximeters) that are secured to a study subject’s preductal site, preferably the right hand, and a postdutcal site, such as any foot, are used with a central aggregator computing device that is able to communicate (wirelessly or otherwise) with the oximeters and store data from the oximeters for eventual retrieval. Measurements may be taken over a predetermined time period, such as three to seven minutes. The subjects may be newborns or infants. Once embodiments of the system (which comprises the oximeters and the computing device) are used to collect data from each newborn or infant in a study cohort, a set of features may be extracted for each subject, such as the min, max, median, variance, mean of each index (PAI, heart rate, SpO2). Subjects in the cohort may be split into training and validation sets (e.g., 80% training vs 20% validation). When using a trained model on a new patient, the input will be the features of the new patient automatically extracted by the model, and the output classification may be, for example, predicted as healthy, CCHD, or non-critical CHD.

In various potential embodiments, data values likely associated with artifact may be removed in pre-processing the data. For example, HR larger than 250 and SpO2 larger than 100 (the pulse oximeter assigns a value of 127 for SpO2 when the measurement quality is poor) may be removed. The minimum, maximum, median, variance, mean of each index (PAI, heart rate, SpO2) may be computed separately, as the features that will be inputted into the machine learning system. Data for new patients will have the same format suited to the machine learning system, such as a comma-separated values (csv) file containing raw data for PPG waveform, SpO2, heart rate, and PAI or PIx.

In various potential embodiments, feature selection may be automated by using, for example, Recursive Feature Elimination (RFE). RFE can help determine the best performance of each model and the corresponding optimal feature set. RFE achieves this by searching for the most relevant subset of features to optimize a performance metric. Cross-validation may be used to optimize sensitivity by setting sensitivity as the score of RFE. In various embodiments, sensitivity may be selected as the score rather than specificity, for example, so as to improve the sensitivity. Additionally, in various embodiments, thresholds may be weighted to optimize specificity. For example, the thresholds between newborns with and without critical congenital heart disease (CCHD) ranges from 0 to 1. The default is 0.5, which means if the model’s prediction for CCHD is greater than 0.5, then the final classification is determined CCHD. To optimize sensitivity using thresholds, decreasing from 0.5 increases sensitivity. To optimize specificity using thresholds, the threshold is increased from 0.5. To achieve the most optimal sensitivity, the process may start with all features from the training dataset as the input and fit the ML models, which ranks features by importance, discards the least important features, and refits the model. This process may be repeated until a desired number of features resulted in the highest sensitivity. The desired number of features may be determined based on the highest sensitivity achieved (see, e.g., FIGS. 6A and 6B).

In various potential embodiments, multiple models (each associated with a different feature set) may be produced based on subject characteristics. For example, one model may be for newborns 0-48 hours in age, and another model may be for newborns/infants 48 hours or older. In certain embodiments, one model may be developed, with, for example, age as a feature in training the model and selecting features. In certain embodiments, an ensemble model that includes, for example, both logistic regression and random forest classifier, as one technique may perform better than another depending on the age of the patient. Different models may also be generated based on whether the presence of a condition is to be detected, or a type (e.g., severity) of vascular disease. Based on an example analysis, the minimum number of features differs by age. In certain example embodiments, the minimum number of features for newborns under 48 hours is 11 features, and the minimum number of features for newborns over 48 hours old is 7 features. In such embodiments, the minimum features to include are SpO2, heart rate, and PAI or PIx. The manner in which those features are included (e.g., min, max, mean, median, or variance) may vary. Additionally, certain models may incorporate radiofemoral delay as a feature.

As will be further discussed below, various embodiments provide a unique approach to the acquisition, structuring, and use of particular data, and identification of optimal combinations of features to detect CCHD or other vascular conditions. Advantageously, integration of multiple factors enhances the ability to detect CCHD. Various embodiments integrate dual right hand and foot measurements of multiple factors (e.g., 5 minutes of heart rate, perfusion index, waveforms and SpO2) with an ML-based approach to provide a significant enhancement over conventional systems (e.g., systems providing a single spot check of oxygenation saturation).

In various potential embodiments, the data acquisition, storage and processing disclosed herein may be applied to non-infant subjects and patients (such as adults, children, etc.). Potential embodiments may use a sensor that fits on adult (or child) fingers and toes, but would be used to collect the same data as for infants. The ML techniques disclosed herein may be applied to identify the unique combination of features suited to detecting vascular conditions in adults. In various embodiments, SpO2 would not be expected to be a predominant feature whereas it is in newborns with CCHD, as newborns with CCHD have a patent ductus arteriosus or defects that allow for right to left shunting of deoxygenated blood. In various embodiments, for adult vascular disease such as aneurysms, aortic dissection, atherosclerosis, peripheral artery disease, vascular graft monitoring and critical limb threatening ischemia, the features of perfusion may relatively be more prominent (e.g., PIx or PAI, radiofemoral delay, and PPG slope) potentially in addition to heart rate.

### BRIEF DESCRIPTION OF THE DRAWINGS

FIG. 1: Illustration of potential uses for this system in post-delivery critical congenital heart disease (CCHD) screening according to various embodiments. Two pulse oximeter devices are applied to both a foot as the postductal site and to a preductal site, preferably the right hand, of a neonate. In an example, Nonin® WristOx2™ 3150 may be used to communicate wirelessly with a computing device that servers as a central aggregator device. The aggregator can then perform visualization and analytics on whether the neonate displays risk for CCHD while simultaneously storing the data for later review.

FIG. 2A: Data collection workflow according to various potential embodiments. Illustration of the workflow of the system. A technician may control the software and attach the pulse oximeters to the patient. They then enter the patient identification number and other medical details. The software will automatically connect to the oximeters via Bluetooth or other wireless communication protocol(s), display the oximetry and perfusion data in real time, and store the data.

FIG. 2B: Example of real-time data visualization during data collection, according to various potential embodiments. During data collection, both the right hand and foot photoplethysmography waveforms along with oxygen saturation (SpO2) and perfusion amplitude index (PAI) values may be displayed on the computing device screen to aid with data quality control.

FIG. 3: Examples of features that can be extracted from raw waveform, according to various potential embodiments. Features that can be extracted from raw waveform include pulse amplitude index (PAI) (FIG. 3A), heart rate including heart rate variability (FIG. 3A), radiofemoral delay (f-h TD) (FIG. 3B), and both the systolic rise and diastolic fall slope of the photoplethysmography waveform (FIG. 3C).

FIG. 4: Example of pulse oximetry data collected from a healthy newborn and a newborn with critical coarctation of the aorta (CoA) according to various potential embodiments. Solid lines are from raw data. Dashed lines have a filtered applied to assist with peak identification due to the dicrotic notch interfering with peak identification for the infant with coarctation. FIG. 4A: A normal newborn demonstrates minimal time delay between the right hand and foot pulse (f-h TD) and similar pulse amplitude index (PAI) in hand and foot. FIG. 4B (10 hours off prostaglandin E1) and FIG. 4C (36 hours off prostaglandin E1): A newborn with critical CoA shows the foot PAI decrease and the f-h TD change as more time off prostaglandin E1 passes. Additionally, the dicrotic notch appearance in the right hand is notable different in the baby with CoA compared to both the healthy newborn and the earlier measurement in the same baby when the ductus arteriosus was presumably more open.

FIG. 5: Feature Analysis between Healthy vs CCHD Over 48 Hour of Age according to various potential embodiments, with FIG. 5A representing Mean SpO2 and FIG. 5B representing to Mean SpO2 and Min HR.

FIG. 6: Recursive Feature Elimination (RFE) by using Machine Learning Models for Healthy vs CCHD at Different Ages according to various potential embodiments, with FIG. 6A representing machine learning models for 0-48 Hour, and FIG. 6B representing machine learning models for over 48 hours.

FIG. 7: Area Under the Receiver Operating Curves (AUROC) for Models on No-CHD vs CCHD according to various potential embodiments, with FIG. 7A representing 0 48 hours and FIG. 7B representing over 48 hours.

FIG. 8: A random forest decision tree simulating the combined oxygen saturation SpO2), heart rate (HR) and pulse amplitude index (PAI) and determination of healthy (no congenital heart disease) vs critical congenital heart disease (CCHD) in newborns using a machine learning model according to various potential embodiments.

FIG. 9: A flow diagram simulating the results of combined oxygenation saturation and perfusion index screening according to various potential embodiments. Patients with systemic obstruction defects are in bold italics; stars (*) indicate patients with critical congenital heart defect requiring intervention in the first month after birth. Abbreviations: AS = aortic stenosis, CHD = congenital heart disease, CoA = coarctation of aorta, DORV = double outlet right ventricle, HLHS = hypoplastic left heart syndrome, IAA = interrupted aortic arch, IVS = intact ventricular septum, MAPCAs = major aortopulmonary collateral arteries, MV = mitral valve, PA = pulmonary atresia, PHTN = pulmonary hypertension, PIx = perfusion index, PS = pulmonary stenosis, SpO₂ = oxygen saturation, TGA = transposition of the great arteries, TOF = Tetralogy of Fallot, VSD = ventricular septal defect.

FIG. 10: Perfusion index (PIx) is a non-invasive measurement of pulsatile blood flow independent of oxygenation that can be measured simultaneously with SpO2 according to various potential embodiments.

FIG. 11: Simultaneous upper and lower extremity pulse oximetry with automated results according to various potential embodiments.

FIG. 12: Example random forest model which may be employed in training a predictive model according to various potential embodiments.

FIG. 13A is a block diagram depicting an embodiment of a network environment comprising a client device in communication with server device.

FIG. 13B is a block diagram depicting a cloud computing environment comprising client device in communication with cloud service providers.

FIGS. 13C - 13D are block diagrams depicting embodiments of computing devices useful in connection with the methods and systems described herein.

FIG. 14 illustrates a system including one or more computing devices, detection devices, and a subject platform according to various potential embodiments.

FIG. 15 shows a flowchart for an example process employing a machine learning approach according to various potential embodiments.

The foregoing and other features of the present disclosure will become apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

### DETAILED DESCRIPTION

In the following detailed description, reference is made to the accompanying drawings, which form a part hereof. The illustrative embodiments described in the detailed description, drawings, and claims are not meant to be limiting. Other embodiments may be utilized, and other changes may be made, without departing from the spirit or scope of the subject matter presented here. It will be readily understood that the aspects of the present disclosure, as generally described herein, and illustrated in the figures, may be arranged, substituted, combined, and designed in a wide variety of different configurations, all of which are explicitly contemplated and make part of this disclosure.

For purposes of reading the description of the various embodiments below, the following descriptions of the sections of the specification and their respective contents may be helpful:
Section A describes systems and methods for acquiring physiological readings used in various potential embodiments
Section B describes embodiments of systems and methods for recognition of vascular conditions via artificial intelligence and machine learning techniques; and
Section C describes a computing environment which may be useful for practicing embodiments described herein.

### Section A: Potential Systems and Methods for Acquiring Physiological Readings

Access to patient medical data is critical to building a real-time data analytic pipeline for improving care providers’ ability to detect, diagnose, and prognosticate diseases. Critical congenital heart disease (CCHD) is a common group of neonatal life-threatening defects that must be promptly diagnosed to minimize morbidity and mortality. CCHD can be diagnosed both prenatally and postnatally. However, despite current screening practices involving oxygen-saturation analysis, timely diagnosis is missed in approximately 900 infants with CCHD annually in the United States (US) and can benefit from increased data processing capabilities. Adding non-invasive perfusion measurements to oxygen-saturation data can improve timeliness and fidelity of CCHD diagnostics. However, suitable real-time monitoring and interpretation of non-invasive perfusion data has been unavailable.

To address this challenge, potential embodiments provide a hardware and software architecture utilizing a computing device for collecting, visualizing, and storing dual oxygen-saturation, perfusion indices, and photoplethysmography data. Data aggregation in the disclosed approach may be automated and data files may be coded with unique study identifiers to facilitate studies.

Using an example system, an example embodiment collected data from 375 neonates, 251 presumably without and 124 with congenital heart disease, in total comprising estimated 3,750 minutes of information. From these data, the example embodiment enabled extraction of non-invasive perfusion features such as perfusion index, radiofemoral delay, and slope of systolic rise or diastolic fall. The disclosed data collection and waveform analysis may be used to enhance CCHD screening algorithms as further discussed below.

To improve CCHD detection and minimize false negative screens, especially of defects associated with poor perfusion such as CoA, non-invasive pulse oximetry measurements such as perfusion index, radiofemoral pulse delay, and other photoplethysmography waveform characteristics may be added to an oxygen-saturation CCHD screening algorithm. However, accessing and interpreting these additional pulse oximetry data is not a prevalent practice. In addition, pulse oximetry devices currently only store de-identified data, limiting its utility for research. In part due to these limitations, prior studies evaluating perfusion index have either included brief (10 second) clinician interpreted and manually documented perfusion indices or have utilized retrospective de-identified samplings from pulse oximetry devices from routine screening.

To solve this problem, various potential embodiments provide an automated real-time data collection and analysis architecture that is able to perform non-invasive measurement of perfusion and oxygenation data. The system is able to wirelessly communicate with pulse oximetry devices and store data on secure computing systems. Potential embodiments provide for a unique data collection pipeline that is customizable by providers depending on the target clinical outcomes. Conventional research or clinical tools are not capable of prospectively collecting abundant high-fidelity CCHD screening data. The insights driven by this data collection will be beneficial to developing algorithmic CCHD screening processes and may be applied to other vascular disease processes that could benefit from non-invasive pulse oximetry perfusion diagnostics (e.g., aneurysms, aortic dissections, atherosclerosis, thrombosis or vascular graft monitoring).

The primary focus of one example study was to explore correlations between oxygen saturation and perfusion data in neonates with and without CCHD. The example study utilized an embodiment comprising an integrative software and hardware system to collect necessary physiologic data. The system: 1) enabled continuous and automated data collection and storage from multiple pulse oximeters simultaneously; 2) ensured accurate time stamping of the collected data; and 3) allowed ease of use by non-technical end users in terms of both data acquisition hardware and data management software.

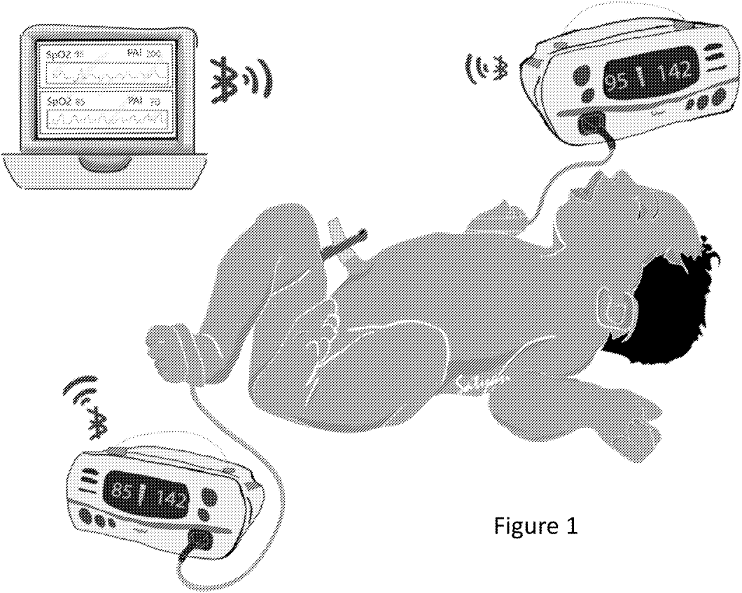

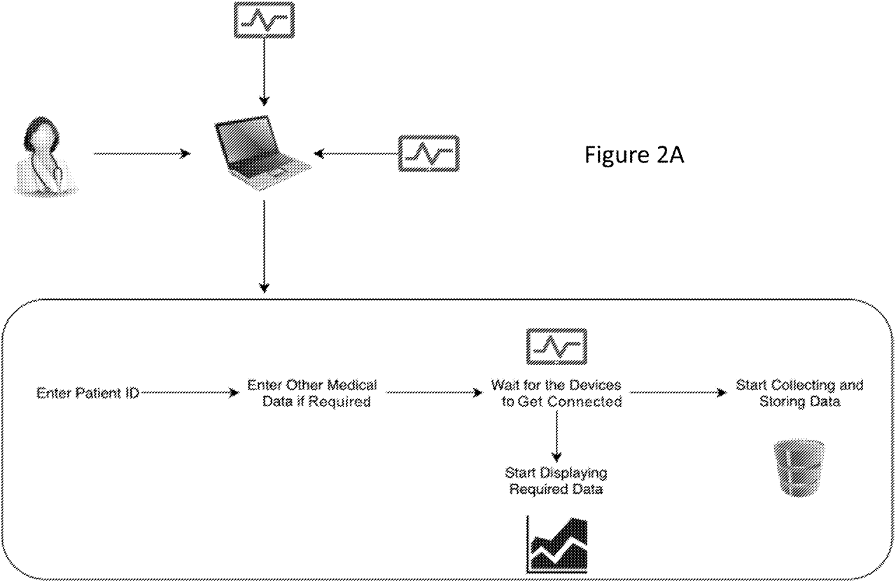

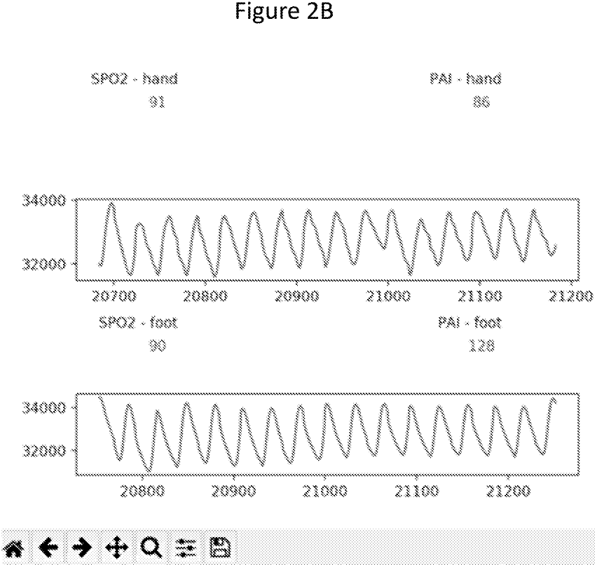

Defects such as CoA are associated with differences in oxygen saturation and perfusion index from the right upper extremity (preductal) and any lower extremity (postductal). Conventional CCHD screening methods employ sequential application of pulse oximetry probe to the right upper extremity followed by a lower extremity with manual documentation and interpretation of data. In various potential embodiments, simultaneous collection of data using dual pulse oximetry (FIG. 1) enables accurate collection and interpretation of data using a simple workflow (FIG. 2A and Table 1).

TABLE 1

Raw data fields collected by example system

| | |
|---|---|
| Data Fields | Explanation |
| Patient_ID | Corresponding patient research identification number |
| Time_Stamp | Time at which the data was recorded |
| FiO₂ | Value of fractional inspired oxygen (FiO₂) as entered by the clinician |
| Pleth | Value of photoplethysmography as received from the Nonin® |
| Measurement Label | Alphanumeric label assigned to the data to easily distinguish consecutive repeat measurements |
| PAI | Pulse Amplitude Index as measured by the Nonin® |
| SpO₂ | Oxygen saturation (SpO₂) as measured by the Nonin® |
| Heart_Rate | Heart Rate of the patient as measured by the Nonin® |

An example system includes two independent perfusion and oxygenation monitors (oximeters) that are attached to the study subject’s preductal site, preferably the right hand, and a postductal site, any foot, and a central aggregator device that is able to communicate with the oximeters and temporarily store their data for eventual retrieval. The example system used, for the oximeter, the Nonin® WristOx2™ 3150. This device allows simultaneous collection of photoplethysmography and oxygenation and perfusion data, can externally transmit data using Bluetooth or other wireless communication protocol, and is relatively inexpensive. The example system used the Pi-top™ computing device, a laptop computer that uses Raspberry Pi microcomputers, as a central aggregator, although any other suitable computing device may be used. By developing appropriate software, example embodiments enable collection of oxygenation and perfusion data via a wireless protocol such as Bluetooth or Wi-Fi (and/or alternative wireless or wired communication methods discussed herein) through, for example, the Nonin®devices. The Pi-top™ is also able to maintain temporal alignment between the two separate Nonin®devices. This helped ensure that data is accurately time-stamped and synchronized. Time stamps can also be adjusted to ensure complete de-identification. A representation of the example system is illustrated in FIG. 1.

Once collected and dated, data files may be automatically aggregated and coded with unique study identifiers. The data may then be uploaded to REDCap or otherwise transferred through a secure data transfer mode. This architecture and workflow (FIG. 2) enable ease of data collection and data sharing across different hospitals.

### Section A1. Data Collection

Using this example system, data from 190 newborns were gathered. At time of enrollment, 130 were presumed to be without congenital heart disease and 60 were expected to have congenital heart disease. In total, the example embodiment gathered approximately 1,665 minutes of pulse oximetry data from these babies in this study. The example embodiment confirmed that 60 of the newborns enrolled as presumably healthy newborns remained healthy. One baby enrolled as a presumably healthy baby before qualifying for the routine CCHD screen, was determined to have CCHD based on the first study measurements collected (oxygen saturations were in the 60s). The follow up is still pending for the remaining babies enrolled as presumably healthy. Of the babies enrolled presumably with congenital heart disease, the example embodiment confirmed the final classification for 48 (31 = CCHD, 13 non-critical CHD and 4 without CHD). In total this comprises 400 minutes of oximetry data from newborns with final classification of congenital heart disease (300 minutes = CCHD, 100 minutes = non-critical CHD) and 610 minutes of oximetry data from newborns confirmed to be without congenital heart disease.

The detailed process of the data collection using this example system is as follows: example embodiments attach two Nonin® WristOx2™ 3150 pulse oximeters to the subject, one on the right hand and the other on either foot. Once these Nonin® devices display valid measurements, and example embodiments can ensure minimal signal noise resulting from limb movement, example embodiments initialize collection software on the Pi-top™ device. In the software user interface, a unique study identification number is entered. This study identifier is an anonymous token that can only be linked back to the patient via an encrypted database like REDCap. Next, example embodiments input the fraction of inspired oxygen (FiO2) the patient is receiving. For study protocols that involve repeat measurements, example embodiments included an alphanumeric entry to label specific measurements for later identification. The software connects to both pulse oximeters via Bluetooth and starts collecting pulse oximetry, oxygen saturation, heart rate, pulse amplitude index (PAI)-synonymous with perfusion index, and photoplethysmography data. All data are time stamped so example embodiments can maintain temporal accuracy between the two Nonin® devices. During collection, data is also displayed on the Pi-top screen in real-time, which aids with data quality control (shown in FIG. 2B). As an illustration of the process in the study, the steps may comprise: (1) turning on Pi-Top (or other computing device); (2) enter password; (3) execute script or otherwise launch application; (4) enter unique identification code, which may include an identifier classifying a subject (e.g., a unique identification ending in “X” may correspond to “Cardiac Babies” and “N” may correspond to “Normal Babies”); (5) input the fraction of inspired oxygen (FiO₂), such as 21 percent; (6) input the alphanumeric label as specified by the study protocol to tag the data with this code; (7) attach the pulse oximeter probes onto the subject’s right hand and the subject’s foot, and connect the respective oximeters to the probes; (8) once the oxygen saturation (SpO₂) on the Nonin® oximeters has stabilized, press start to start simultaneous data collection from the two oximeters; (9) check to see that both the oximeters have started Bluetooth or other wireless transmission (e.g., through a visual indicator confirming that wireless transmission is enabled); (10) the computing device should start displaying waveforms for the hand and foot (see, e.g., FIG. 2B); and (11) stop data collection through the computing device (e.g., by shutting down the Pi-Top using the power button).

### Section A2. Data Aggregation

Management of each computing device, including data aggregation, may be performed by a technician or research coordinator present on the site. The technician may 1) upload data to the REDCap database, 2) back up the folders in the computing device (e.g., to an external hard drive), and 3) delete all the files on the computing device when the data is stored locally somewhere. Uploaded data can then be accessed across the research network and referenced by patient identifier.

### Section A3. Scalability and Adding New Devices

Provisioning additional computing devices and pulse oximeter devices enables the collection of multiple patients simultaneously and prevents data collection slowdowns in study progress due to lost or damaged equipment. To more rapidly provision devices, example embodiments employed an image of the computing device with the pre-configured details of the software that can be copied to a storage medium (e.g., the microSD card of the Pi-top) in a single step resulting in a fully functional computing device without the need to install multiple software components. This workflow substantially decreases the number of steps necessary for computing device deployment and may allow non-technical users to assist more experienced personnel in the provisioning process, which may be successfully done remotely with sites when a software change is needed.

### Section A4. Data Collected and Feature Identification

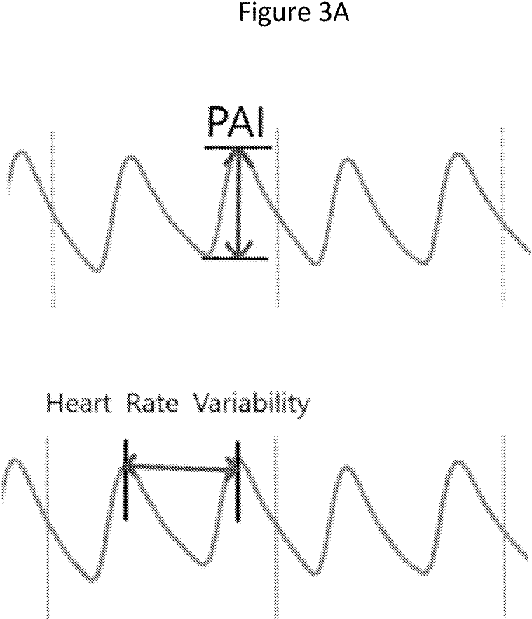

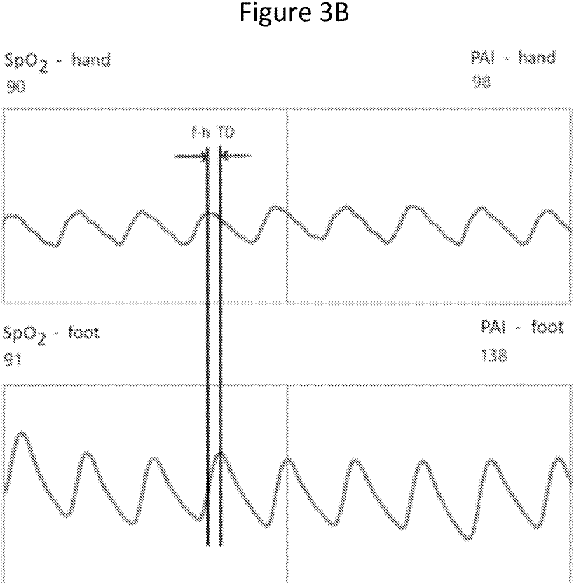

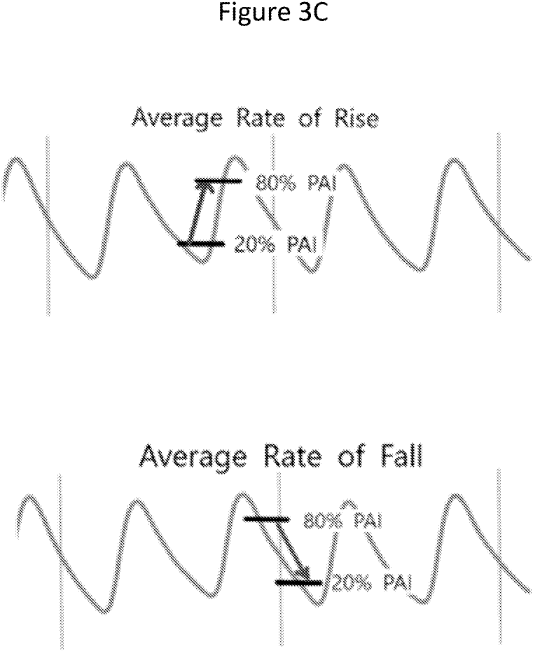

Table 1 summarizes the data fields collected from the Nonin® devices for each patient enrolled. The data are visualized on the Pi-top (FIG. 2B) for real-time interpretation and then can be reconstructed from the logged data for analysis and feature detection on a compute server. From the collected data, example embodiments enabled extraction of different features stated in Table 2. For example, FIGS. 3B and 3C illustrate how the delays between systolic peaks of hand and foot waveforms (Feature #6 in Table 2) and the slopes of systolic rise and diastolic fall (Feature #3 and #4 in Table 2) can be extracted from reconstructed waveforms. Various statistics of these features (minimum, maximum, mean, standard deviation, median) may be useful for further characterization and development of prediction model for CCHD detection.

TABLE 2

Features that can be extracted from the data fields collected

| Feature Number | Features |
|---|---|
| 1 | Systolic and Diastolic peaks |
| 2 | Perfusion Index |
| 3 | Slope of systolic rise |
| 4 | Slope of diastolic fall |
| 5 | Area under the curve per pulse |
| 6 | Delay between systolic maximum points of hand vs. foot waveforms |
| 7 | Delay between systolic starting points of hand vs. foot waveforms |
| 8 | Oxygen saturation over a defined period of time |
| 9 | Heart rate |

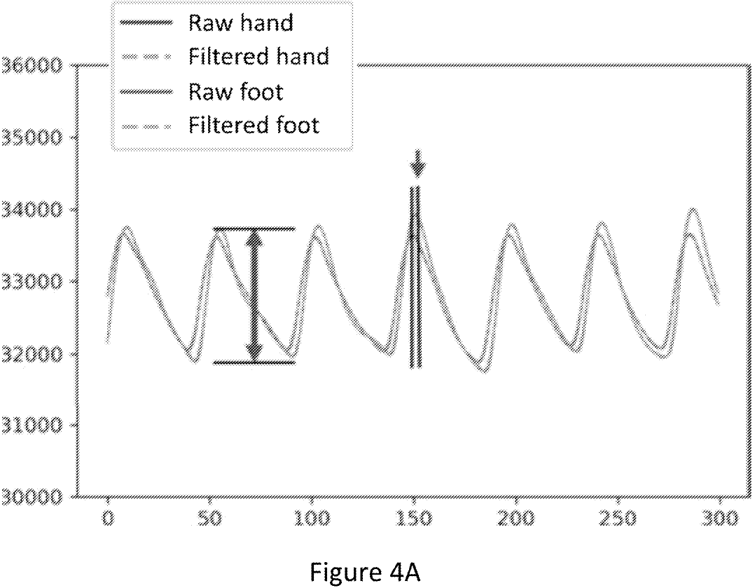

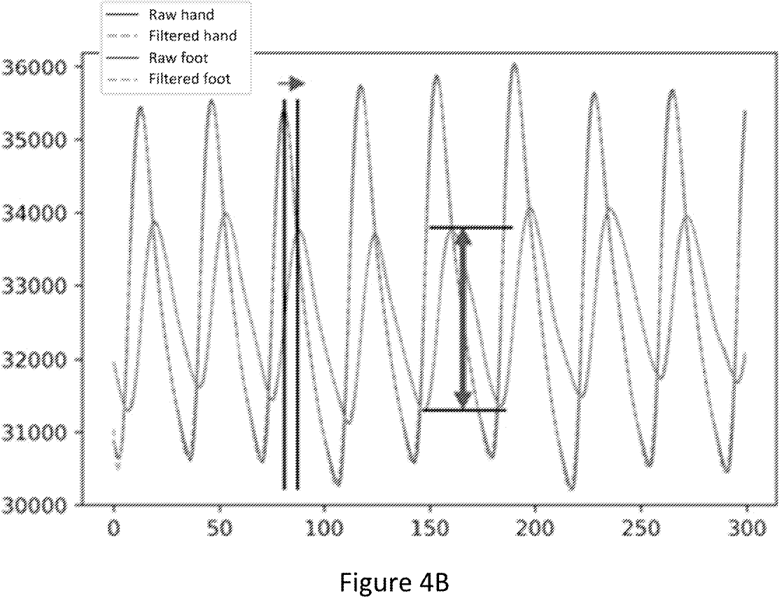

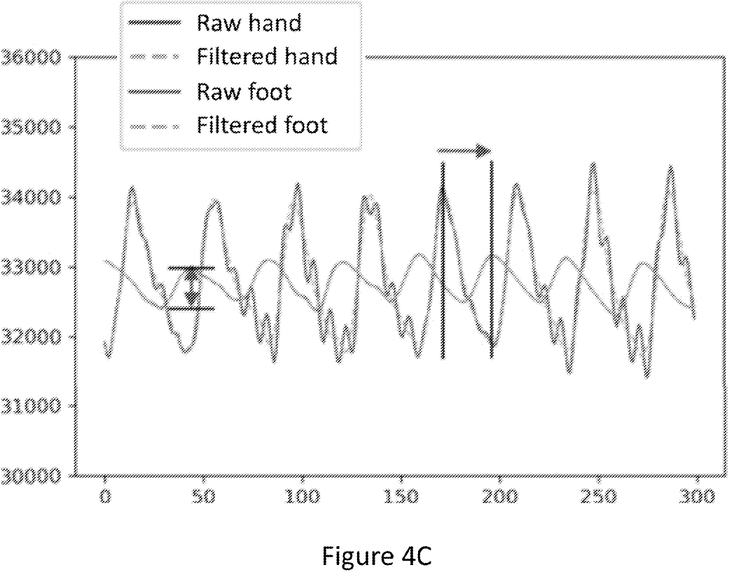

To further demonstrate the features collected in the data, example embodiments included photoplethysmography waveforms from a healthy baby (FIG. 4A) and a baby with critical CoA (FIGS. 4B and 4C). Interestingly, the baby with CoA was trialed off prostaglandin E1 therapy to assess the severity of the coarctation and thus the ductus arteriosus was presumably closing and the CoA narrowing during data collection. Measurements occurred approximately 10 hours and 36 hours after the prostaglandin E1 infusion was discontinued. The baby with CoA was monitored in the neonatal intensive care unit and was asymptomatic. The baby had an echocardiogram at 3 days of age, which noted the ductus arteriosus was closed and the CoA was minimal. Thus, the baby was discharged home with plans to follow up outpatient with cardiology. The baby then represented to care at 7 days of age, and an echocardiogram showed an increase in the gradient across the CoA prompting readmission and subsequent surgery for CoA repair. Note the foot pulse amplitude index (PAI) decreased in this neonate with increasing duration off prostaglandin E1 infusion. The average foot PAI decreased from 96 to 36 during this time period (FIGS. 4B and 4C).

There are some other notable features in this baby with CoA compared with the healthy baby (FIGS. 4A - 4C). Note the closer proximity of the peaks of the hand and foot (f-hTD) in the healthy baby versus the baby with CoA. Additionally, note how the f-hTD changes as more hours off prostaglandin pass and presumably as the ductus arteriosus closes. In various embodiments, if the computing device (such as the Pi-Top™) can only receive one Bluetooth (or other wireless communication protocol) data package at a time, there may be a transmission difference between the hand and foot waveforms that should to be accounted for to more accurately quantify f-hTD. Accordingly, in alternative embodiments, a computing device capable of receiving multiple wireless transmission data packages at a time may be employed (e.g., one with multiple transceivers capable of working independently), or multiple computing devices may be used to receive time-stamped readings from the oximeters (e.g., one computing device for each oximeter), and the readings from the multiple computing devices may be combined (e.g., by one of the multiple computing devices being used as a data aggregator, or by another computing device that receives readings therefrom) to obtain f-hTD.

Additionally, as a baby’s physiology changes and the potential for physiological foot peaks occurring before the hand peaks, this further complicates the analysis. However, the photoplethysmography segments shown in FIGS. 4A and 4B, were chosen because the relative hand to foot peak durations were consistent for these measurements. Thus, assuming a similar wireless transmission across patients, this highlights the potential for a longer f-hTD in patients with CoA. Example embodiments also captured a morphology change to the hand photoplethysmography waveform as the ductus arteriosus closed with the development of a remarkably notable dicrotic notch (FIG. 4C). This highlighted the need to apply smoothing filters to the waveforms so that peaks can more easily be identified to ease analysis.

### Section A5. Challenges and Example Adjustments That May Be Made

To enhance data security, an example embodiment may employ, for example, an encrypted USB compatible with Linux operating system and functional on an ARM processor (Raspberry Pi). Such an embodiment may require using VeraCrypt™ encryption service as opposed to an off-the-shelf USB.

Additionally, to ensure cessation of the Bluetooth connection in the example embodiment discussed, the entire Pi-top™ system may need to be shut down. If the Bluetooth connection remains, then the data collection can continue when not intended. This may be problematic if data collection has to be restarted. For example, if a technical error is encountered mid data collection, then the system has to be powered down and restarted.

To better identify collected data with specific clinical scenarios, such as age of the patient at time of the measurement, an alphanumeric entry that serves as a code specific to the protocol time points may be employed. This may be useful if, for example, the time-stamps on the collected data may not be used (for additional security and more complete de-identification of the data). It is noted, however, that time-stamps alone may not be sufficient for identification due to time drift caused by not having the Pi-top™ connected to a server for security purposes.

### Section A6: Potential Embodiments With Variations

Various embodiments may provide a system for automated collection of pulse oximetry data for research related to CCHD screening. This system collects and displays real-time data from medical devices while communicating wirelessly (via, e.g., Bluetooth). The system does not require access to other networks, making it portable to the regions with little or no access to the internet and also aids with security. It is inexpensive and capable of end-to-end automation of data collection and storage. The system requires only one clinician or coordinator with basic computer skills to monitor the process. Once it is set up properly, the system can capture oxygen saturation and perfusion data from the infant in a non-invasive manner, and is able to maintain continuous operation for as long as required by the study circumstances.

Embodiments of the disclosed data collection system may be motivated by the need for early CCHD detection, and the inability for providers to electronically store pulse oximetry data in a manner conducive to research and clinical needs. Antenatal echocardiology and postnatal examination detected only approximately 70% of the patients with CCHD leading to the addition of postnatal pulse oximetry to routine newborn screening. Techniques disclosed herein address the limitations of the oxygen-saturation based CCHD screen. Adding non-invasive pulse oximetry measurements such as perfusion index (PIx), radiofemoral pulse delay (f-hTD), and other photoplethysmography waveform characteristics to the current diagnostic suite of pulse oximetry measurement may help in more accurate detection of CCHD in patients. For example, in a prior study with a total of 123 normal and 21 newborns with congenital heart defects, four out of five critical systemic obstruction subjects passed the regular oxygen-saturation based CCHD screen. The addition of PIx to the screen resulted in four of the five newborns failing the combined screen. That study and prior studies evaluated PIx for a single moment in time with an artifact free waveform for 10 seconds. In the example study disclosed herein, the pulse amplitude index (PAI), which is PIx multiplied by 100, was averaged over 5 minutes. Example embodiments enabled captured a patient with CoA and demonstrated the foot PAI decrease, as the ductus arteriosus was closing, and decreased below thresholds that may be indicative of systemic obstruction such as CoA. Interestingly, this decrease in PAI was noted when the echocardiogram estimated only a mild CoA and thus it was initially thought the baby may not require surgery. Therefore, additional non-invasive perfusion measurements along with the regular oxygen-saturation based screen is a potential technique to not only enhance CCHD detection, but may also help guide treatment plans when it is already known that a patient has a CoA. Additionally, although prior studies have noted prolonged pulse delay and abnormalities in timing of pre-ductal dichroitic notch in newborns with CoA, the data provided herein captured an identifiable pre-ductal dicrotic notch while an infant with CoA was trialed off prostaglandin therapy and the timing relationship between the hand and foot pulses also appeared to change as well.

Future research into CCHD screening techniques will necessitate readily accessible medical device data, data standards, and archival data storage of identifiable data (identifiable at least through unique identification codes). Use of pulse oximetry devices results in huge amounts of data that are yet to be fully explored for CCHD screening. The current infrastructure limits access to this data for research. For example, continuous pulse oximetry monitoring in the hospital may not be stored long term or be readily identifiable, and when it is, researchers have to rely on charting to correlate with clinically relevant data such as probe placement. The disclosed pulse oximeter data collection process mitigates many of these barriers and may also help guide future screenings or research for other diseases that may benefit from non-invasive pulse oximetry perfusion diagnostics (aneurysms, aortic dissections, atherosclerosis, thrombosis, or vascular graft monitoring).

Although certain embodiments disclosed herein involved collection of pulse oximetry and perfusion information from a single type of medical device (Nonin® WristOx2™ 3150) using Bluetooth, other devices, and other communication protocols besides Bluetooth, may be employed in various other potential embodiments. Embodiments of the disclosed system provide for collection of data from subjects (e.g., neonates) who have already undergone or will undergo standard of care CCHD screening by a qualified provider to generate labeled datasets, and such data may be employed to develop advanced machine learning based CCHD detection models as further discussed below. In various potential embodiments, analytic capabilities may be incorporated into individual data streams such as perfusion. For example, this streaming waveform data may be employed in an analytic capacity and possibly even perform CCHD screening interpretation.

Overall, embodiments of the disclosed approach open up a new way of data collection for CCHD related diagnostic information from neonates or other subjects. The data collection system and process may be automated and capable of being used by non-technical users. It may also serve as a generalized model to be utilized by different researchers who are also working to collect streaming waveform information. Embodiments of the disclosed system gathered approximately 3,750 minutes of oximetry data from neonates with and without congenital heart disease. This information may be invaluable for developing future CCHD screening processes and hopefully will serve as the basis for future electronic systems to improve CCHD detection and may be adapted for other research endeavors as well.

### Section B: Various Potential Embodiments of Systems and Methods for Recognition Of Vascular Conditions via Artificial Intelligence and Machine Learning Techniques

As indicated above, CCHD screening that only uses oxygen saturation, measured by pulse oximetry, fails to detect an estimated 900 US newborns annually. The addition of other pulse oximetry features such as perfusion index, heart rate, pulse delay and photoplethysmography characteristics, however, can improve detection of CCHD, especially those with systemic blood flow obstruction such as Coarctation of the Aorta (CoA). To comprehensively study the most relevant features associated with CCHD, example embodiments investigated interpretable machine learning (ML) algorithms by using Recursive Feature Elimination (RFE) to identify an optimal subset of features. Example embodiments incorporated the trained ML models into the current SpO2-alone screening algorithm. Example embodiments of the disclosed enhanced CCHD screening system, which adds the ML model, improved sensitivity by approximately 10 percentage points compared to the current standard SpO2-alone method with minimal to no impact on specificity. Embodiments of the disclosed ML approach combine pulse oximetry features to improve detection of CCHD with little impact on false positive rate.

Congenital heart disease (CHD) is the most common birth defect, and critical congenital heart disease (CCHD) is a subset of CHD accounting for almost 20% of these infants, representing the most severe forms of CHD. CCHD lesions require surgical or catheter-based intervention soon after birth, often including pre-procedural hospitalization and medical management. Late or missed detection of CCHD can lead to significant, preventable morbidity, as well as death. The majority of the missed types of CCHD defects are those with obstructed systemic blood flow that do not commonly cause low SpO2, or hypoxemia.

Various embodiments of the disclosed approach address this problem by providing an automated real-time data collection system to collect additional pulse oximetry data in newborns, allowing us to analyze other pulse oximetry features that may augment the current screening process when added to the SpO2 screening component. As such, it is necessary to design an interpretable machine learning model that can be directly incorporated into example embodiments of the SpO2-alone screening system with automatic feature selection to further improve the sensitivity of CCHD detection with little impact on specificity (at least 99%). Embodiments thus analyze the feature relevance of CCHD screening by using machine learning (ML) algorithms and incorporate ML into current standard SpO2 screening.

### Section B1: Data Collection of Subjects

In example embodiments, by using an automated collection system, 335 newborns were enrolled, including 236 newborns that have a final diagnosis (with or without CCHD) confirmed. Patients were excluded if they required vasoactive infusions other than Prostaglandin E1. Example embodiments recorded at least 5-minute dual limb (right hand and any foot) pulse oximetry measurements at three time periods: within 24 hours, 24 - 48 hours, and after 48 hours following the baby’s birth. For the following analysis, healthy newborns (defined as those without any CHD) vs those with CCHD (newborns who require a surgical or catheter-based intervention within 30 days of age) were analyzed. Example embodiments divided all measurements into two groups: (G1) 0-48 hours, which included 158 healthy and 27 CCHD newborns; and (G2) over 48 hours, which included 50 healthy and 36 CCHD newborns.

### Section B2: Spot SpO2-Alone Screening

Single pre- and post-ductal SpO2 values were recorded during the measurements as well. These spot values were used to assign pass or fail to all newborns per the current standard SpO2-alone screen. If the last recorded spot SpO2 measurements resulted in a “repeat” assignment from this algorithm, example embodiments assigned them a “fail” in a “Conservative spot SpO2-alone” algorithm to bias towards the null for CCHD detection.

### Section B3: Features Extraction & Analysis

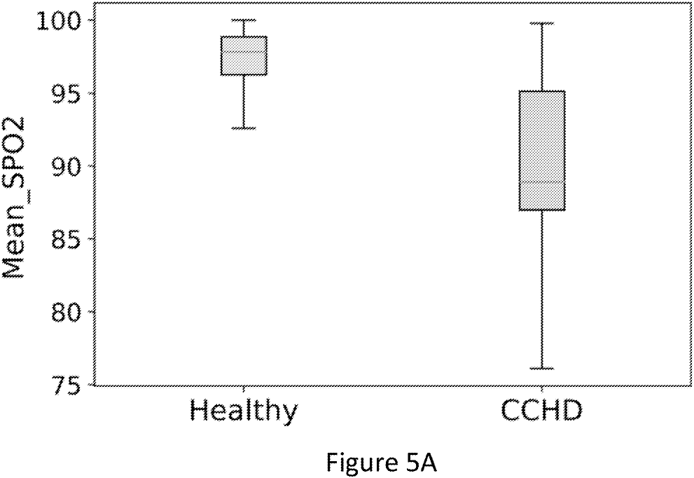

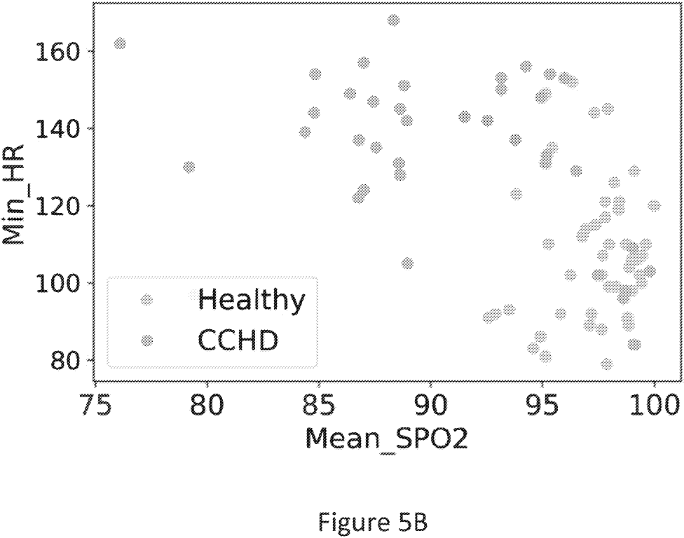

Pulse oximetry features evaluated for discrimination of healthy vs CCHD include: heart rate (HR), perfusion amplitude index (PAI), also known as perfusion index (PIx), and oxygen saturation (SpO2). Example embodiments removed values likely associated with artifact: HR larger than 250 and SpO2 larger than 100 (the pulse oximeter assigns a value of 127 for SpO2 when the measurement quality is poor). Example embodiments then extracted variance, min, max, median and mean for HR, PAI and SpO2. To study the differentiation of these features, example embodiments visualized each individually and then their correlation with each other. FIGS. 5A and 5B illustrate the distribution of the mean SpO2 and its correlation with min HR: the mean SpO2 (FIG. 5A) for the healthy newborns is typically higher than that for the CCHD newborns and the min HR (FIG. 5B) for healthy newborns is typically less than that for CCHD newborns.

### Section B4. Classification Algorithms

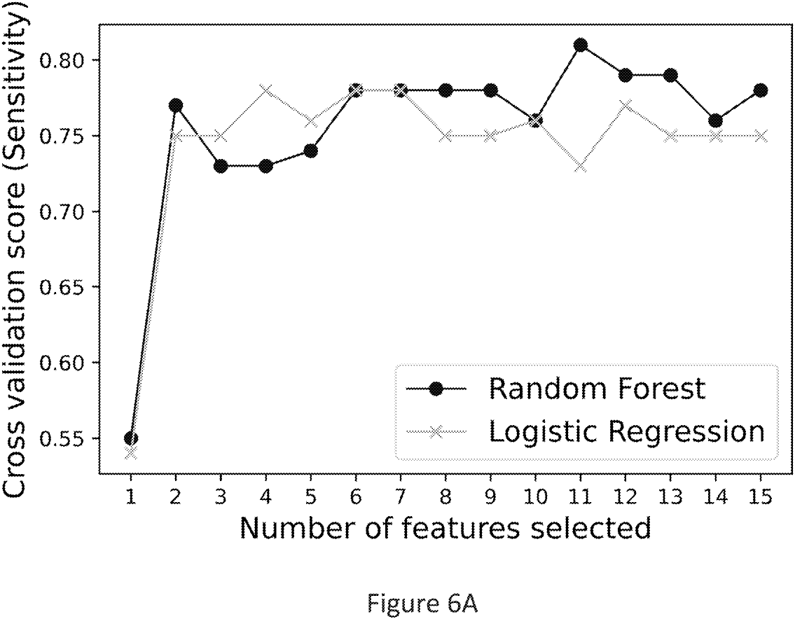

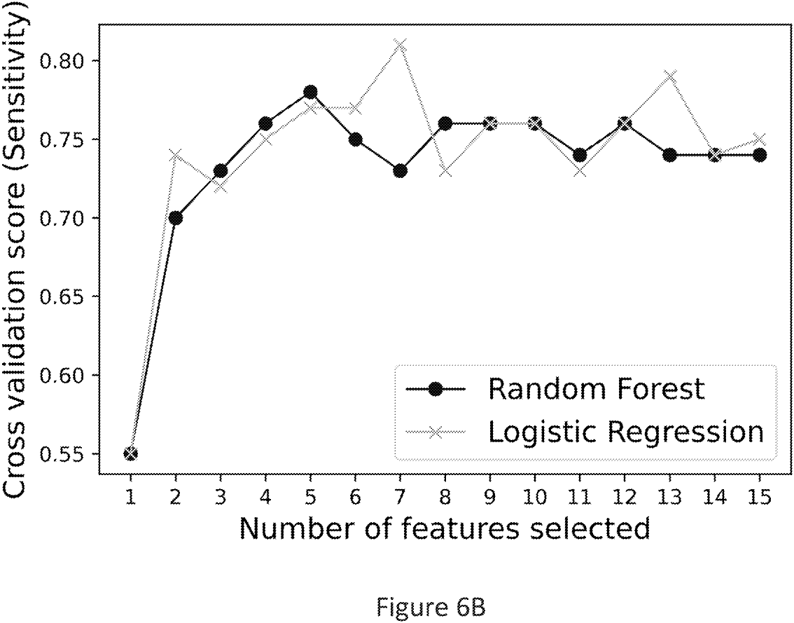

Several ML classifiers were tested for CCHD detection during this study, including Random Forest, Logistic Regression, and Multilayer Perceptron. Example embodiments comprehensively investigated the above classification algorithms by Recursive Feature Elimination (RFE) with 5-fold cross-validation on each algorithm separately. RFE can help determine the best performance of each model and the corresponding optimal feature set. RFE achieves this by searching for the most relevant subset of features to optimize the performance metric. Example embodiments used cross-validation to optimize sensitivity by setting it as the score of RFE. To achieve the most optimal sensitivity, example embodiments started with all features from the training dataset as the input and fit the ML models, which ranked features by importance, discarded the least important features and refit the model. This process was repeated until the desired number of features resulted in the highest sensitivity (as shown in FIGS. 6A and 6B).

1) 0-48 Hours of age to evaluate for no-CHD vs CCHD: In this setting, example embodiments found that Random Forest Classifier resulted in the highest sensitivity in the 5-fold cross-validation for the 0-48 hour age. FIG. 6A shows the optimal sensitivity was achieved by using 11 features: median HR, mean HR, maximum HR, HR variance, minimum SpO2, maximum SpO2, median SpO2, mean SpO2, mean PAI (or PIx), median PAI (or PIx), and maximum PAI (or PIx).

2) Over 48 Hours of age to evaluate for no-CHD vs CCHD: example embodiments found that Logistic Regression performed best in this group, as FIG. 6B demonstrates, example embodiments found a subset of 7 optimal features using Logistic Regression: minimum HR, maximum HR, HR variance, median SpO2, mean SpO2, mean PAI (or PIx), and minimum PAI (or PIx).

The fewer number of CCHD cases compared to healthy cases resulted in a class imbalance problem, making it challenging to train a relatively unbiased ML model. To deal with this issue, we applied Random Oversampling (ROS), Random Undersampling (RUS), and Synthetic Minority Oversampling Technique (SMOTE), separately. SMOTE is one type of oversampling method, where the minority class is oversampled by generating “composite” examples. We tried different sampling ratios from 0.2 to 0.9, but the results had trivial improvements. Thus, we used the balanced loss function inside of the ML models, which led to the optimal results we could achieve in this study.

### Section B5: Performance Evaluation

In various embodiments, CCHD screening may be a binary classification problem between healthy and CCHD, thus we used the following metrics to comprehensively evaluate the performance of the model: Sensitivity (Sens) (1) and Specificity (Spec) (2).

$$\text{Sens = TPR =} \frac{\text{TP}}{\text{TP} + \text{FN}} \qquad \text{(1)}$$

$$\text{Spec =} \frac{\text{TP}}{\text{TN} + \text{FP}} \qquad \text{(2)}$$

$$\text{FPR =} \frac{F P}{F P + T N} \qquad \text{(3)}$$

where:
TP: the number of CCHD predicted as CCHD
FP: the number of healthy predicted as CCHD
TN: the number of healthy predicted as healthy
FN: the number of CCHD predicted as healthy

Example embodiments also calculated the Area Under the Receiver Operating Characteristics curve (AUROC) by plotting true positive rate (TPR) (1) against false positive rate (FPR) (3) with the discrimination threshold increasing from 0 to 1.

### Section B6: Sensitivity and Specificity Results

The sensitivity and specificity of embodiments of the disclosed approach and the current SpO2 screening methods are summarized in Table 3. When comparing to the current standard CCHD screening, which includes spot right hand and any foot SpO2 measurements, the addition of the over 48 hour ML model did not lead to additional false positive results, but did detect 4 additional newborns with CCHD, hence increasing sensitivity from 76.5% to 88.9% (McNemar mid-p p=0.06) when tested on a sample of 50 healthy newborns and 36 newborns with CCHD. The addition of the 0-48 hour ML model, resulted in 3 additional false positive results and detected 3 additional newborns with CCHD increasing sensitivity from 76.5% to 85.2% (McNemar mid-p p=0.13) when tested on a sample of 158 healthy newborns and 27 newborns with CCHD. Overall, embodiments of the disclosed ML models improved the sensitivity from 76.5% to 86.4%, a nearly 10 percentage point improvement.

TABLE 3

Compared with current spo2 screening

| Methods | Specificity | Sensitivity |
|---|---|---|
| True Spot SpO2-alone | 96.8 | 62.8 |
| Conservative Spot SpO2-alone | 96.8 | 76.5 |
| ML (0-48 hrs) | 97.5 | 81.5 |
| ML (>48 hrs) | 100 | 83.3 |
| Conservative Spot SpO2-alone + ML (0-48 hrs) | 96.2 | 85.2 |
| Conservative Spot SpO2-alone + ML (>48 hrs) | 96 | 88.9 |
| Conservative Spot SpO2-alone + Any ML | 95.8 | 86.4 |

### Section B7: ROC Curve

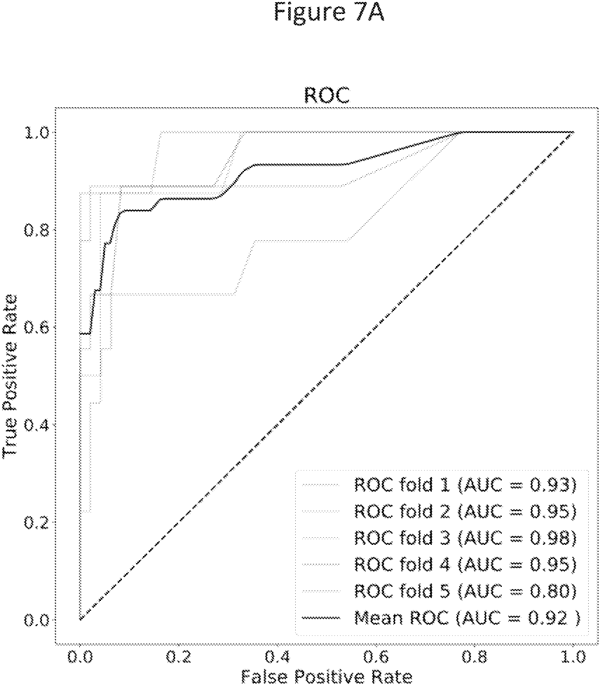

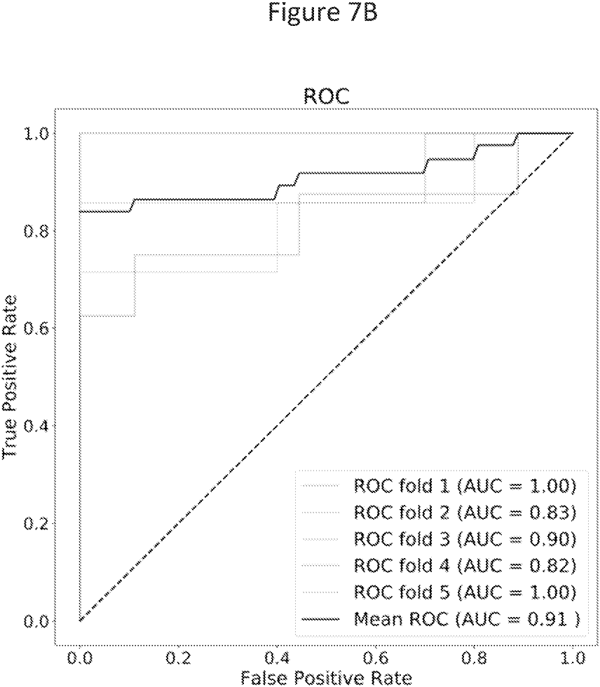

ROC curve results from 5-fold testing are shown in FIG. 7. For 0-48 hours (FIG. 7A), the average AUROC for no-CHD vs CCHD was 0.92 by using the Random Forest classifier; for over 48 hours (FIG. 7B), the average AUROC was 0.91 by using the Logistic Regression model. The estimated AUROC for example ML algorithms combining pulse oximetry features (PAI and HR) appears similar to or better than the current SpO2-alone screen.

### Section B8: Interpretable Machine Learning

Compared to the current standard SpO2-alone CCHD screening, disclosed embodiments of the ML models can achieve better sensitivity by incorporating features related to HR and PAI (or PIx). The optimal subset for the 0-48 hours Random Forest classifier includes: HR (median, mean, max, variance), SpO2 (min, max, median, mean), PAI or PIx (mean, median, max). The optimal subset for the over 48 hours Logistic Regression ML model includes: HR (min, max, variance), SpO2 (median, mean), PAI or PIx (mean, min). Therefore, in example embodiments, the features extracted from HR and PAI have potentials for CCHD detection.

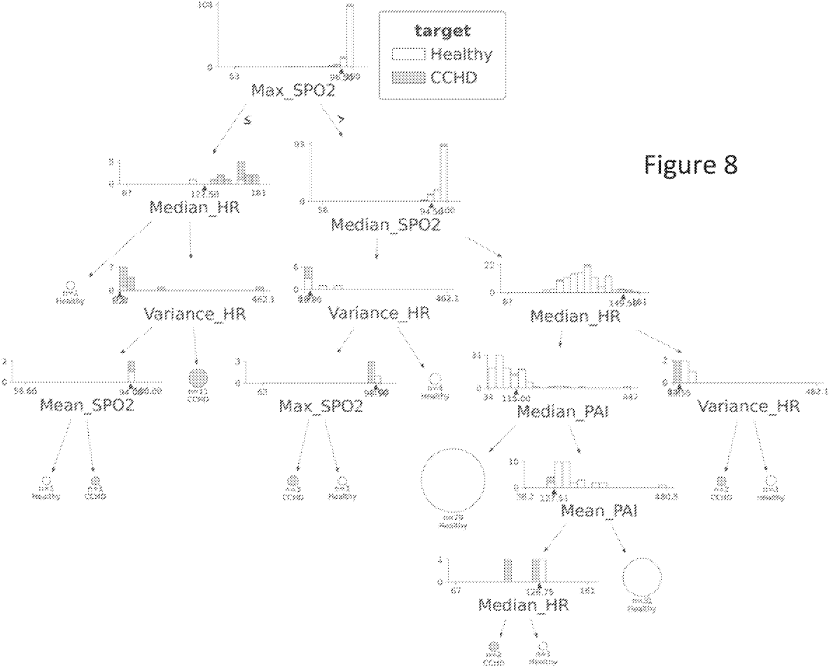

In various embodiments, from Logistic Regression, example embodiments achieved importance ranking of features based on the trained weights. For the Random Forest classifier, example embodiments visualized decision trees as shown in FIG. 8: the decision tree mainly relies on the features from SpO2, but the features related to HR and PAI are also considered in deeper decision-making layers of the tree.

As discussed above, various embodiments employ ML techniques with optimized feature selection to provide an enhanced CCHD screening algorithm. Example embodiments first applied the current standard CCHD screening (including both True Spot SpO2-alone and Conservative Spot SpO2-alone) to enrolled newborns as the benchmark. Then, example embodiments tested example embodiments of ML models on these newborns, which gained approximately 10 percentage points increase in sensitivity of CCHD detection. Embodiments of the disclosed system improve detection of defects currently missed by SpO2-alone which would serve as a promising enhanced CCHD screening tool. Furthermore, in various embodiments, by using these interpretable ML models, example embodiments found potential benefit of PAI and HR as features to differentiate between healthy newborns and newborns with CCHD. In various embodiments, to further improve the sensitivity, other potential features related to CCHD diagnosis such as radiofemoral pulse delay and photoplethysmography slopes may be incorporated.

### Section C: Systems, Devices, and Methods for Machine Learning Modeling

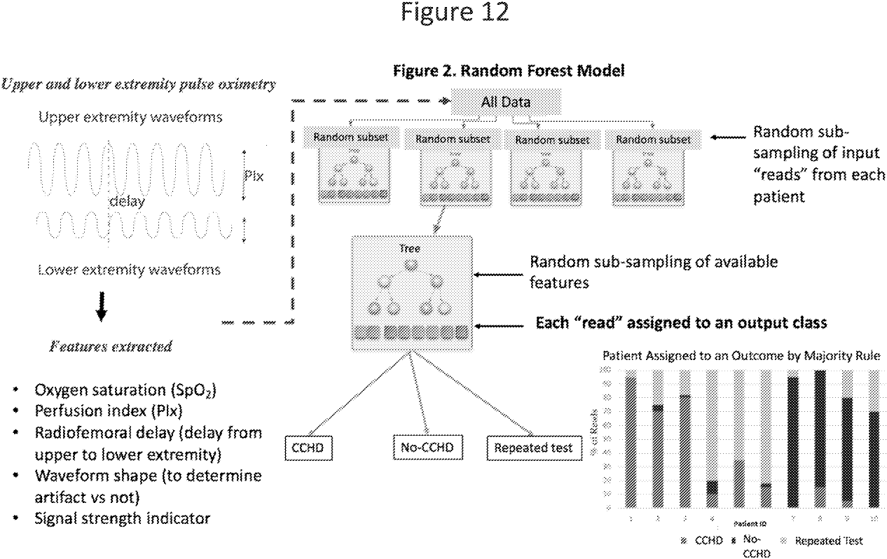

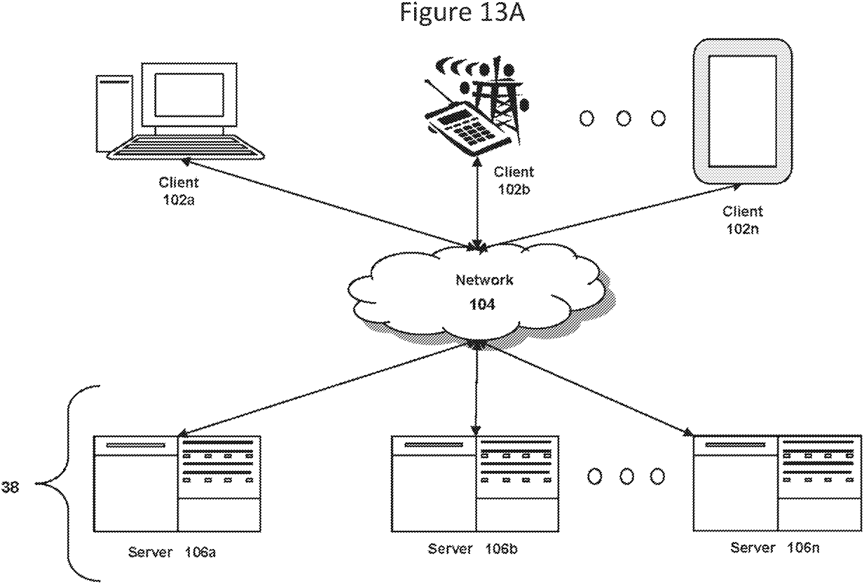

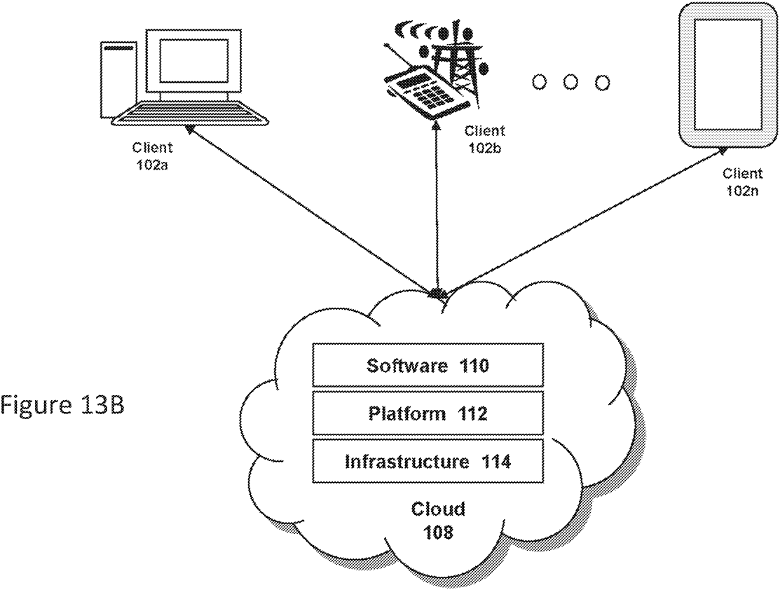

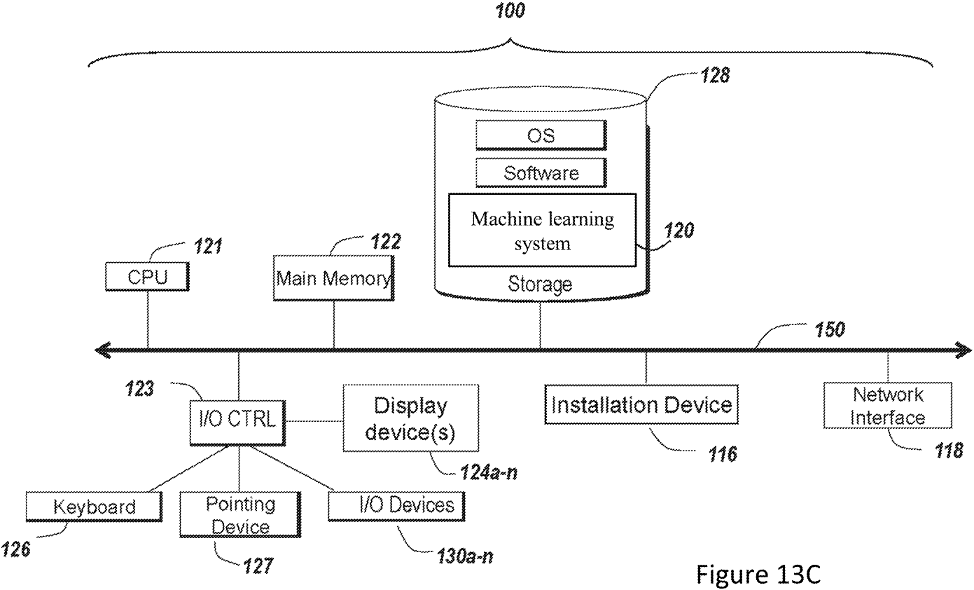

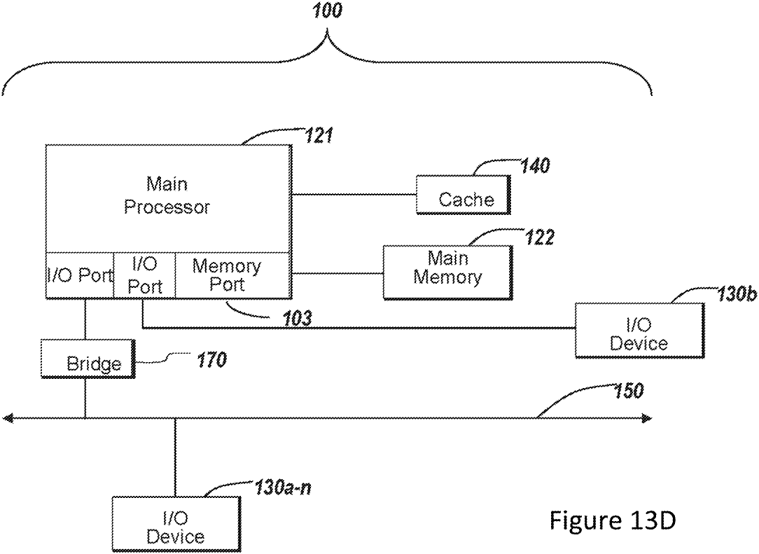

Aspects of the operating environment as well as associated system components (e.g., hardware elements) in connection with various embodiments of the methods and systems described herein will now be discussed. Referring to FIG. 13A, an embodiment of a network environment is depicted. In brief overview, the network environment includes one or more clients 102a-102n (also generally referred to as local machine(s) 102, client(s) 102, client node(s) 102, client machine(s) 102, client computer(s) 102, client device(s) 102, endpoint(s) 102, or endpoint node(s) 102) in communication with one or more servers 106a-106n (also generally referred to as server(s) 106, node 106, or remote machine(s) 106) via one or more networks 104. In some embodiments, a client 102 has the capacity to function as both a client node seeking access to resources provided by a server and as a server providing access to hosted resources for other clients 102a-102n.

Although FIG. 13A shows a network 104 between the clients 102 and the servers 106, the clients 102 and the servers 106 may be on the same network 104. In some embodiments, there are multiple networks 104 between the clients 102 and the servers 106. In one of these embodiments, a network 104′ (not shown) may be a private network and a network 104 may be a public network. In another of these embodiments, a network 104 may be a private network and a network 104′ a public network. In still another of these embodiments, networks 104 and 104′ may both be private networks.

The network 104 may be connected via wired or wireless links. Wired links may include Digital Subscriber Line (DSL), coaxial cable lines, or optical fiber lines. The wireless links may include BLUETOOTH, Wi-Fi, Worldwide Interoperability for Microwave Access (WiMAX), an infrared channel or satellite band. The wireless links may also include any cellular network standards used to communicate among mobile devices, including standards that qualify as 1G, 2G, 3G, 4G, or 5G. The network standards may qualify as one or more generation of mobile telecommunication standards by fulfilling a specification or standards such as the specifications maintained by International Telecommunication Union. The 3G standards, for example, may correspond to the International Mobile Telecommunications-2000 (IMT-2000) specification, and the 4G standards may correspond to the International Mobile Telecommunications Advanced (IMT-Advanced) specification. Examples of cellular network standards include AMPS, GSM, GPRS, UMTS, LTE, LTE Advanced, Mobile WiMAX, and WiMAX-Advanced. Cellular network standards may use various channel access methods e.g. FDMA, TDMA, CDMA, or SDMA. In some embodiments, different types of data may be transmitted via different links and standards. In other embodiments, the same types of data may be transmitted via different links and standards.

The network 104 may be any type and/or form of network. The geographical scope of the network 104 may vary widely and the network 104 can be a body area network (BAN), a personal area network (PAN), a local-area network (LAN), e.g. Intranet, a metropolitan area network (MAN), a wide area network (WAN), or the Internet. The topology of the network 104 may be of any form and may include, e.g., any of the following: point-to-point, bus, star, ring, mesh, or tree. The network 104 may be an overlay network which is virtual and sits on top of one or more layers of other networks 104′. The network 104 may be of any such network topology as known to those ordinarily skilled in the art capable of supporting the operations described herein. The network 104 may utilize different techniques and layers or stacks of protocols, including, e.g., the Ethernet protocol, the internet protocol suite (TCP/IP), the ATM (Asynchronous Transfer Mode) technique, the SONET (Synchronous Optical Networking) protocol, or the SDH (Synchronous Digital Hierarchy) protocol. The TCP/IP internet protocol suite may include application layer, transport layer, internet layer (including, e.g., IPv6), or the link layer. The network 104 may be a type of a broadcast network, a telecommunications network, a data communication network, or a computer network.

In some embodiments, the system may include multiple, logically-grouped servers 106. In one of these embodiments, the logical group of servers may be referred to as a server farm 38 or a machine farm 38. In another of these embodiments, the servers 106 may be geographically dispersed. In other embodiments, a machine farm 38 may be administered as a single entity. In still other embodiments, the machine farm 38 includes a plurality of machine farms 38. The servers 106 within each machine farm 38 can be heterogeneous - one or more of the servers 106 or machines 106 can operate according to one type of operating system platform (e.g., WINDOWS NT, manufactured by Microsoft Corp. of Redmond, Washington), while one or more of the other servers 106 can operate on according to another type of operating system platform (e.g., Unix, Linux, or Mac OS X).

In one embodiment, servers 106 in the machine farm 38 may be stored in high-density rack systems, along with associated storage systems, and located in an enterprise data center. In this embodiment, consolidating the servers 106 in this way may improve system manageability, data security, the physical security of the system, and system performance by locating servers 106 and high performance storage systems on localized high performance networks. Centralizing the servers 106 and storage systems and coupling them with advanced system management tools allows more efficient use of server resources.

The servers 106 of each machine farm 38 do not need to be physically proximate to another server 106 in the same machine farm 38. Thus, the group of servers 106 logically grouped as a machine farm 38 may be interconnected using a wide-area network (WAN) connection or a metropolitan-area network (MAN) connection. For example, a machine farm 38 may include servers 106 physically located in different continents or different regions of a continent, country, state, city, campus, or room. Data transmission speeds between servers 106 in the machine farm 38 can be increased if the servers 106 are connected using a local-area network (LAN) connection or some form of direct connection. Additionally, a heterogeneous machine farm 38 may include one or more servers 106 operating according to a type of operating system, while one or more other servers 106 execute one or more types of hypervisors rather than operating systems. In these embodiments, hypervisors may be used to emulate virtual hardware, partition physical hardware, virtualize physical hardware, and execute virtual machines that provide access to computing environments, allowing multiple operating systems to run concurrently on a host computer. Native hypervisors may run directly on the host computer. Hypervisors may include VMware ESX/ESXi, manufactured by VMWare, Inc., of Palo Alto, California; the Xen hypervisor, an open source product whose development is overseen by Citrix Systems, Inc.; the HYPER-V hypervisors provided by Microsoft or others. Hosted hypervisors may run within an operating system on a second software level. Examples of hosted hypervisors may include VMware Workstation and VIRTUALBOX.

Management of the machine farm 38 may be de-centralized. For example, one or more servers 106 may comprise components, subsystems and modules to support one or more management services for the machine farm 38. In one of these embodiments, one or more servers 106 provide functionality for management of dynamic data, including techniques for handling failover, data replication, and increasing the robustness of the machine farm 38. Each server 106 may communicate with a persistent store and, in some embodiments, with a dynamic store.

Server 106 may be a file server, application server, web server, proxy server, appliance, network appliance, gateway, gateway server, virtualization server, deployment server, SSL VPN server, or firewall. In one embodiment, the server 106 may be referred to as a remote machine or a node. In another embodiment, a plurality of nodes 290 may be in the path between any two communicating servers.

Referring to FIG. 13B, a cloud computing environment is depicted. A cloud computing environment may provide client 102 with one or more resources provided by a network environment. The cloud computing environment may include one or more clients 102a-102n, in communication with the cloud 108 over one or more networks 104. Clients 102 may include, e.g., thick clients, thin clients, and zero clients. A thick client may provide at least some functionality even when disconnected from the cloud 108 or servers 106. A thin client or a zero client may depend on the connection to the cloud 108 or server 106 to provide functionality. A zero client may depend on the cloud 108 or other networks 104 or servers 106 to retrieve operating system data for the client device. The cloud 108 may include back end platforms, e.g., servers 106, storage, server farms or data centers.

The cloud 108 may be public, private, or hybrid. Public clouds may include public servers 106 that are maintained by third parties to the clients 102 or the owners of the clients. The servers 106 may be located off-site in remote geographical locations as disclosed above or otherwise. Public clouds may be connected to the servers 106 over a public network. Private clouds may include private servers 106 that are physically maintained by clients 102 or owners of clients. Private clouds may be connected to the servers 106 over a private network 104. Hybrid clouds 108 may include both the private and public networks 104 and servers 106.

The cloud 108 may also include a cloud based delivery, e.g. Software as a Service (SaaS) 110, Platform as a Service (PaaS) 112, and Infrastructure as a Service (IaaS) 114. IaaS may refer to a user renting the use of infrastructure resources that are needed during a specified time period. IaaS providers may offer storage, networking, servers or virtualization resources from large pools, allowing the users to quickly scale up by accessing more resources as needed. Examples of IaaS can include infrastructure and services (e.g., EG-32) provided by OVH HOSTING of Montreal, Quebec, Canada, AMAZON WEB SERVICES provided by Amazon.com, Inc., of Seattle, Washington, RACKSPACE CLOUD provided by Rackspace US, Inc., of San Antonio, Texas, Google Compute Engine provided by Google Inc. of Mountain View, California, or RIGHTSCALE provided by RightScale, Inc., of Santa Barbara, California. PaaS providers may offer functionality provided by IaaS, including, e.g., storage, networking, servers or virtualization, as well as additional resources such as, e.g., the operating system, middleware, or runtime resources. Examples of PaaS include WINDOWS AZURE provided by Microsoft Corporation of Redmond, Washington, Google App Engine provided by Google Inc., and HEROKU provided by Heroku, Inc. of San Francisco, California. SaaS providers may offer the resources that PaaS provides, including storage, networking, servers, virtualization, operating system, middleware, or runtime resources. In some embodiments, SaaS providers may offer additional resources including, e.g., data and application resources. Examples of SaaS include GOOGLE APPS provided by Google Inc., SALESFORCE provided by Salesforce.com Inc. of San Francisco, California, or OFFICE 365 provided by Microsoft Corporation. Examples of SaaS may also include data storage providers, e.g. DROPBOX provided by Dropbox, Inc. of San Francisco, California, Microsoft SKYDRIVE provided by Microsoft Corporation, Google Drive provided by Google Inc., or Apple ICLOUD provided by Apple Inc. of Cupertino, California.

Clients 102 may access IaaS resources with one or more IaaS standards, including, e.g., Amazon Elastic Compute Cloud (EC2), Open Cloud Computing Interface (OCCI), Cloud Infrastructure Management Interface (CIMI), or OpenStack standards. Some IaaS standards may allow clients access to resources over HTTP, and may use Representational State Transfer (REST) protocol or Simple Object Access Protocol (SOAP). Clients 102 may access PaaS resources with different PaaS interfaces. Some PaaS interfaces use HTTP packages, standard Java APIs, JavaMail API, Java Data Objects (JDO), Java Persistence API (JPA), Python APIs, web integration APIs for different programming languages including, e.g., Rack for Ruby, WSGI for Python, or PSGI for Perl, or other APIs that may be built on REST, HTTP, XML, or other protocols. Clients 102 may access SaaS resources through the use of web-based user interfaces, provided by a web browser (e.g. GOOGLE CHROME, Microsoft INTERNET EXPLORER, or Mozilla Firefox provided by Mozilla Foundation of Mountain View, California). Clients 102 may also access SaaS resources through smartphone or tablet applications, including, e.g., Salesforce Sales Cloud, or Google Drive app. Clients 102 may also access SaaS resources through the client operating system, including, e.g., Windows file system for DROPBOX.

In some embodiments, access to IaaS, PaaS, or SaaS resources may be authenticated. For example, a server or authentication server may authenticate a user via security certificates, HTTPS, or API keys. API keys may include various encryption standards such as, e.g., Advanced Encryption Standard (AES). Data resources may be sent over Transport Layer Security (TLS) or Secure Sockets Layer (SSL).

The client 102 and server 106 may be deployed as and/or executed on any type and form of computing device, e.g. a computer, network device or appliance capable of communicating on any type and form of network and performing the operations described herein. FIGS. 13C and 13D depict block diagrams of a computing device 100 useful for practicing an embodiment of the client 102 or a server 106. As shown in FIGS. 13C and 13D, each computing device 100 includes a central processing unit 121, and a main memory unit 122. As shown in FIG. 13C, a computing device 100 may include a storage device 128, an installation device 116, a network interface 118, an I/O controller 123, display devices 124a-124n, a keyboard 126 and a pointing device 127, e.g. a mouse. The storage device 128 may include, without limitation, an operating system, software, and a software of a machine learning system 120. As shown in FIG. 13D, each computing device 100 may also include additional optional elements, e.g. a memory port 103, a bridge 170, one or more input/output devices 130a-130n (generally referred to using reference numeral 130), and a cache memory 140 in communication with the central processing unit 121.

The central processing unit 121 is any logic circuitry that responds to and processes instructions fetched from the main memory unit 122. In many embodiments, the central processing unit 121 is provided by a microprocessor unit, e.g.: those manufactured by Intel Corporation of Mountain View, California; those manufactured by Motorola Corporation of Schaumburg, Illinois; the ARM processor and TEGRA system on a chip (SoC) manufactured by Nvidia of Santa Clara, California; the POWER7 processor, those manufactured by International Business Machines of White Plains, New York; or those manufactured by Advanced Micro Devices of Sunnyvale, California. The computing device 100 may be based on any of these processors, or any other processor capable of operating as described herein. The central processing unit 121 may utilize instruction level parallelism, thread level parallelism, different levels of cache, and multi-core processors. A multi-core processor may include two or more processing units on a single computing component. Examples of multi-core processors include the AMD PHENOM IIX2, INTEL CORE i5 and INTEL CORE i7.

Main memory unit or memory device 122 may include one or more memory chips capable of storing data and allowing any storage location to be directly accessed by the microprocessor 121. Main memory unit or device 122 may be volatile and faster than storage 128 memory. Main memory units or devices 122 may be Dynamic random access memory (DRAM) or any variants, including static random access memory (SRAM), Burst SRAM or SynchBurst SRAM (BSRAM), Fast Page Mode DRAM (FPM DRAM), Enhanced DRAM (EDRAM), Extended Data Output RAM (EDO RAM), Extended Data Output DRAM (EDO DRAM), Burst Extended Data Output DRAM (BEDO DRAM), Single Data Rate Synchronous DRAM (SDR SDRAM), Double Data Rate SDRAM (DDR SDRAM), Direct Rambus DRAM (DRDRAM), or Extreme Data Rate DRAM (XDR DRAM). In some embodiments, the main memory 122 or the storage 128 may be non-volatile; e.g., non-volatile read access memory (NVRAM), flash memory non-volatile static RAM (nvSRAM), Ferroelectric RAM (FeRAM), Magnetoresistive RAM (MRAM), Phase-change memory (PRAM), conductive-bridging RAM (CBRAM), Silicon-Oxide-Nitride-Oxide-Silicon (SONOS), Resistive RAM (RRAM), Racetrack, Nano-RAM (NRAM), or Millipede memory. The main memory 122 may be based on any of the above described memory chips, or any other available memory chips capable of operating as described herein. In the embodiment shown in FIG. 13C, the processor 121 communicates with main memory 122 via a system bus 150 (described in more detail below). FIG. 13D depicts an embodiment of a computing device 100 in which the processor communicates directly with main memory 122 via a memory port 103. For example, in FIG. 13D the main memory 122 may be DRDRAM.

FIG. 13D depicts an embodiment in which the main processor 121 communicates directly with cache memory 140 via a secondary bus, sometimes referred to as a backside bus. In other embodiments, the main processor 121 communicates with cache memory 140 using the system bus 150. Cache memory 140 typically has a faster response time than main memory 122 and is typically provided by SRAM, BSRAM, or EDRAM. In the embodiment shown in FIG. 13D, the processor 121 communicates with various I/O devices 130 via a local system bus 150. Various buses may be used to connect the central processing unit 121 to any of the I/O devices 130, including a PCI bus, a PCI-X bus, or a PCI-Express bus, or a NuBus. For embodiments in which the I/O device is a video display 124, the processor 121 may use an Advanced Graphics Port (AGP) to communicate with the display 124 or the I/O controller 123 for the display 124. FIG. 13D depicts an embodiment of a computer 100 in which the main processor 121 communicates directly with I/O device 130b or other processors 121′ via HYPERTRANSPORT, RAPIDIO, or INFINIBAND communications technology. FIG. 13D also depicts an embodiment in which local busses and direct communication are mixed: the processor 121 communicates with I/O device 130a using a local interconnect bus while communicating with I/O device 130b directly.

A wide variety of I/O devices 130a-130n may be present in the computing device 100. Input devices may include keyboards, mice, trackpads, trackballs, touchpads, touch mice, multi-touch touchpads and touch mice, microphones, multi-array microphones, drawing tablets, cameras, single-lens reflex camera (SLR), digital SLR (DSLR), CMOS sensors, accelerometers, infrared optical sensors, pressure sensors, magnetometer sensors, angular rate sensors, depth sensors, proximity sensors, ambient light sensors, gyroscopic sensors, or other sensors. Output devices may include video displays, graphical displays, speakers, headphones, inkjet printers, laser printers, and 3D printers.

Devices 130a-130n may include a combination of multiple input or output devices, including, e.g., Microsoft KINECT, Nintendo Wiimote for the WII, Nintendo WII U GAMEPAD, or Apple IPHONE. Some devices 130a-130n allow gesture recognition inputs through combining some of the inputs and outputs. Some devices 130a-130n provides for facial recognition which may be utilized as an input for different purposes including authentication and other commands. Some devices 130a-130n provides for voice recognition and inputs, including, e.g., Microsoft KINECT, SIRI for IPHONE by Apple, Google Now or Google Voice Search.

Additional devices 130a-130n have both input and output capabilities, including, e.g., haptic feedback devices, touchscreen displays, or multi-touch displays. Touchscreen, multi-touch displays, touchpads, touch mice, or other touch sensing devices may use different technologies to sense touch, including, e.g., capacitive, surface capacitive, projected capacitive touch (PCT), in-cell capacitive, resistive, infrared, waveguide, dispersive signal touch (DST), in-cell optical, surface acoustic wave (SAW), bending wave touch (BWT), or force-based sensing technologies. Some multi-touch devices may allow two or more contact points with the surface, allowing advanced functionality including, e.g., pinch, spread, rotate, scroll, or other gestures. Some touchscreen devices, including, e.g., Microsoft PIXELSENSE or Multi-Touch Collaboration Wall, may have larger surfaces, such as on a table-top or on a wall, and may also interact with other electronic devices. Some I/O devices 130a-130n, display devices 124a-124n or group of devices may be augment reality devices. The I/O devices may be controlled by an I/O controller 123 as shown in FIG. 13C. The I/O controller may control one or more I/O devices, such as, e.g., a keyboard 126 and a pointing device 127, e.g., a mouse or optical pen. Furthermore, an I/O device may also provide storage and/or an installation medium 116 for the computing device 100. In still other embodiments, the computing device 100 may provide USB connections (not shown) to receive handheld USB storage devices. In further embodiments, an I/O device 130 may be a bridge between the system bus 150 and an external communication bus, e.g. a USB bus, a SCSI bus, a FireWire bus, an Ethernet bus, a Gigabit Ethernet bus, a Fibre Channel bus, or a Thunderbolt bus.

In some embodiments, display devices 124a-124n may be connected to I/O controller 123. Display devices may include, e.g., liquid crystal displays (LCD), thin film transistor LCD (TFT-LCD), blue phase LCD, electronic papers (e-ink) displays, flexile displays, light emitting diode displays (LED), digital light processing (DLP) displays, liquid crystal on silicon (LCOS) displays, organic light-emitting diode (OLED) displays, active-matrix organic light-emitting diode (AMOLED) displays, liquid crystal laser displays, time-multiplexed optical shutter (TMOS) displays, or 3D displays. Examples of 3D displays may use, e.g. stereoscopy, polarization filters, active shutters, or autostereoscopy. Display devices 124a-124n may also be a head-mounted display (HMD). In some embodiments, display devices 124a-124n or the corresponding I/O controllers 123 may be controlled through or have hardware support for OPENGL or DIRECTX API or other graphics libraries.

In some embodiments, the computing device 100 may include or connect to multiple display devices 124a-124n, which each may be of the same or different type and/or form. As such, any of the I/O devices 130a-130n and/or the I/O controller 123 may include any type and/or form of suitable hardware, software, or combination of hardware and software to support, enable or provide for the connection and use of multiple display devices 124a-124n by the computing device 100. For example, the computing device 100 may include any type and/or form of video adapter, video card, driver, and/or library to interface, communicate, connect or otherwise use the display devices 124a-124n. In one embodiment, a video adapter may include multiple connectors to interface to multiple display devices 124a-124n. In other embodiments, the computing device 100 may include multiple video adapters, with each video adapter connected to one or more of the display devices 124a-124n. In some embodiments, any portion of the operating system of the computing device 100 may be configured for using multiple displays 124a-124n. In other embodiments, one or more of the display devices 124a-124n may be provided by one or more other computing devices 100a or 100b connected to the computing device 100, via the network 104. In some embodiments software may be designed and constructed to use another computer’s display device as a second display device 124a for the computing device 100. For example, in one embodiment, an Apple iPad may connect to a computing device 100 and use the display of the device 100 as an additional display screen that may be used as an extended desktop. One ordinarily skilled in the art will recognize and appreciate the various ways and embodiments that a computing device 100 may be configured to have multiple display devices 124a-124n.

Referring again to FIG. 13C, the computing device 100 may comprise a storage device 128 (e.g. one or more hard disk drives or redundant arrays of independent disks) for storing an operating system or other related software, and for storing application software programs such as any program related to the software for the machine learning system 120 (which may comprise a machine learning modeler). Examples of storage device 128 include, e.g., hard disk drive (HDD); optical drive including CD drive, DVD drive, or BLU-RAY drive; solid-state drive (SSD); USB flash drive; or any other device suitable for storing data. Some storage devices may include multiple volatile and non-volatile memories, including, e.g., solid state hybrid drives that combine hard disks with solid state cache. Some storage device 128 may be non-volatile, mutable, or read-only. Some storage device 128 may be internal and connect to the computing device 100 via a bus 150. Some storage devices 128 may be external and connect to the computing device 100 via an I/O device 130 that provides an external bus. Some storage device 128 may connect to the computing device 100 via the network interface 118 over a network 104, including, e.g., the Remote Disk for MACBOOK AIR by Apple. Some client devices 100 may not require a non-volatile storage device 128 and may be thin clients or zero clients 102. Some storage device 128 may also be used as an installation device 116, and may be suitable for installing software and programs. Additionally, the operating system and the software can be run from a bootable medium, for example, a bootable CD, e.g. KNOPPIX, a bootable CD for GNU/Linux that is available as a GNU/Linux distribution from knoppix.net.

Client device 100 may also install software or application from an application distribution platform. Examples of application distribution platforms include the App Store for iOS provided by Apple, Inc., the Mac App Store provided by Apple, Inc., GOOGLE PLAY for Android OS provided by Google Inc., Chrome Webstore for CHROME OS provided by Google Inc., and Amazon Appstore for Android OS and KINDLE FIRE provided by Amazon.com, Inc. An application distribution platform may facilitate installation of software on a client device 102. An application distribution platform may include a repository of applications on a server 106 or a cloud 108, which the clients 102a-102n may access over a network 104. An application distribution platform may include application developed and provided by various developers. A user of a client device 102 may select, purchase and/or download an application via the application distribution platform.

Furthermore, the computing device 100 may include a network interface 118 to interface to the network 104 through a variety of connections including, but not limited to, standard telephone lines LAN or WAN links (e.g., 802.11, T1, T3, Gigabit Ethernet, Infiniband), broadband connections (e.g., ISDN, Frame Relay, ATM, Gigabit Ethernet, Ethernet-over-SONET, ADSL, VDSL, BPON, GPON, fiber optical including FiOS), wireless connections, or some combination of any or all of the above. Connections can be established using a variety of communication protocols (e.g., TCP/IP, Ethernet, ARCNET, SONET, SDH, Fiber Distributed Data Interface (FDDI), IEEE 802.11a/b/g/n/ac CDMA, GSM, WiMax and direct asynchronous connections). In one embodiment, the computing device 100 communicates with other computing devices 100′ via any type and/or form of gateway or tunneling protocol e.g. Secure Socket Layer (SSL) or Transport Layer Security (TLS), or the Citrix Gateway Protocol manufactured by Citrix Systems, Inc. of Ft. Lauderdale, Florida. The network interface 118 may comprise a built-in network adapter, network interface card, PCMCIA network card, EXPRESSCARD network card, card bus network adapter, wireless network adapter, USB network adapter, modem or any other device suitable for interfacing the computing device 100 to any type of network capable of communication and performing the operations described herein.

A computing device 100 of the sort depicted in FIGS. 13B and 13C may operate under the control of an operating system, which controls scheduling of tasks and access to system resources. The computing device 100 can be running any operating system such as any of the versions of the MICROSOFT WINDOWS operating systems, the different releases of the Unix and Linux operating systems, any version of the MAC OS for Macintosh computers, any embedded operating system, any real-time operating system, any open source operating system, any proprietary operating system, any operating systems for mobile computing devices, or any other operating system capable of running on the computing device and performing the operations described herein. Typical operating systems include, but are not limited to: WINDOWS 2000, WINDOWS Server 2022, WINDOWS CE, WINDOWS Phone, WINDOWS XP, WINDOWS VISTA, and WINDOWS 7, WINDOWS RT, and WINDOWS 8 all of which are manufactured by Microsoft Corporation of Redmond, Washington; MAC OS and iOS, manufactured by Apple, Inc. of Cupertino, California; and Linux, a freely-available operating system, e.g. Linux Mint distribution (“distro”) or Ubuntu, distributed by Canonical Ltd. of London, United Kingdom; or Unix or other Unix-like derivative operating systems; and Android, designed by Google, of Mountain View, California, among others. Some operating systems, including, e.g., the CHROME OS by Google, may be used on zero clients or thin clients, including, e.g., CHROMEBOOKS.

The computer system 100 can be any workstation, telephone, desktop computer, laptop or notebook computer, netbook, ULTRABOOK, tablet, server, handheld computer, mobile telephone, smartphone or other portable telecommunications device, media playing device, a gaming system, mobile computing device, or any other type and/or form of computing, telecommunications or media device that is capable of communication. The computer system 100 has sufficient processor power and memory capacity to perform the operations described herein. The computer system 100 can be of any suitable size, such as a standard desktop computer or a Raspberry Pi 4 manufactured by Raspberry Pi Foundation, of Cambridge, United Kingdom. In some embodiments, the computing device 100 may have different processors, operating systems, and input devices consistent with the device. The Samsung GALAXY smartphones, e.g., operate under the control of Android operating system developed by Google, Inc. GALAXY smartphones receive input via a touch interface.

In some embodiments, the computing device 100 is a gaming system. For example, the computer system 100 may comprise a PLAYSTATION 3, or PERSONAL PLAYSTATION PORTABLE (PSP), or a PLAYSTATION VITA device manufactured by the Sony Corporation of Tokyo, Japan, a NINTENDO DS, NINTENDO 3DS, NINTENDO WII, or a NINTENDO WII U device manufactured by Nintendo Co., Ltd., of Kyoto, Japan, an XBOX 360 device manufactured by the Microsoft Corporation of Redmond, Washington.

In some embodiments, the computing device 100 is a digital audio player such as the Apple IPOD, IPOD Touch, and IPOD NANO lines of devices, manufactured by Apple Computer of Cupertino, California. Some digital audio players may have other functionality, including, e.g., a gaming system or any functionality made available by an application from a digital application distribution platform. For example, the IPOD Touch may access the Apple App Store. In some embodiments, the computing device 100 is a portable media player or digital audio player supporting file formats including, but not limited to, MP3, WAV, M4A/AAC, WMA Protected AAC, AIFF, Audible audiobook, Apple Lossless audio file formats and .mov, .m4v, and .mp4 MPEG-4 (H.264/MPEG-4 AVC) video file formats.

In some embodiments, the computing device 100 is a tablet e.g. the IPAD line of devices by Apple; GALAXY TAB family of devices by Samsung; or KINDLE FIRE, by Amazon.com, Inc. of Seattle, Washington. In other embodiments, the computing device 100 is an eBook reader, e.g. the KINDLE family of devices by Amazon.com, or NOOK family of devices by Barnes & Noble, Inc. of New York City, New York.

In some embodiments, the communications device 102 includes a combination of devices, e.g. a smartphone combined with a digital audio player or portable media player. For example, one of these embodiments is a smartphone, e.g. the IPHONE family of smartphones manufactured by Apple, Inc.; a Samsung GALAXY family of smartphones manufactured by Samsung, Inc.; or a Motorola DROID family of smartphones. In yet another embodiment, the communications device 102 is a laptop or desktop computer equipped with a web browser and a microphone and speaker system, e.g. a telephony headset. In these embodiments, the communications devices 102 are web-enabled and can receive and initiate phone calls. In some embodiments, a laptop or desktop computer is also equipped with a webcam or other video capture device that enables video chat and video call.

In some embodiments, the status of one or more machines 102, 106 in the network 104 are monitored, generally as part of network management. In one of these embodiments, the status of a machine may include an identification of load information (e.g., the number of processes on the machine, CPU and memory utilization), of port information (e.g., the number of available communication ports and the port addresses), or of session status (e.g., the duration and type of processes, and whether a process is active or idle). In another of these embodiments, this information may be identified by a plurality of metrics, and the plurality of metrics can be applied at least in part towards decisions in load distribution, network traffic management, and network failure recovery as well as any aspects of operations of the present solution described herein. Aspects of the operating environments and components described above will become apparent in the context of the systems and methods disclosed herein.

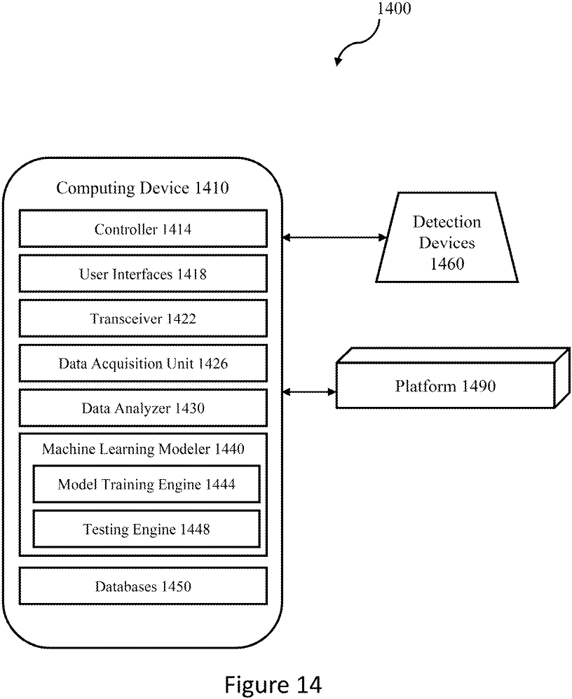

Referring to FIG. 14, in various embodiments, an example system 1400 may include a computing device 1410 (or multiple computing devices, co-located or remote to each other) communicatively coupled to on which a subject may be situated, detection devices 1460, and a platform 1490. In various embodiments, computing device 1410 (or components thereof) may be integrated with the detection devices 1460 (or components thereof), which may include, for example, one or more pulse oximeters and/or other sensors that may sense physiological data from the subject on the platform 1490. The computing device 1410 may be or may include a data aggregator computing device as disclosed herein. The computing device 1410 may also be, or may include, a machine learning system (comprising, for example, components discussed herein such as the machine learning modeler 1440). Components of computing device 1410 may be implemented by various combinations of computing hardware and software.

The computing device 1410 may include a controller 1414 having one or more processors and one or more volatile and non-volatile memories for storing computing code executable by the one or more processors, and data and/or signals that are captured, acquired, recorded, and/or generated via, for example, detection devices 1460. The controller 1414 may control, directly or indirectly, various components of computing device 1410, detection devices 1460, and/or platform 1490. The controller 1414 may be configured to exchange control signals with detection devices 1460 and/or the platform 1490, allowing the computing device 1410 to be used to control, for example, the acquisition of physiological readings and/or delivery of data generated and/or acquired through the detection devices 1460.

Computing device 1410 may include a data acquisition unit 1426 that may be configured to exchange control signals with detection devices 1460 (or components thereof), allowing the computing device 1410 to be used to control the capture of physiological data and/or signals via sensors of the detection devices 1460, retrieve data or signals (e.g., from detection devices 1460 and/or memory devices where data is stored), and direct to transfer of data or signals (e.g., to detection devices 1460 as feedback thereto, to memory for storage, and/or to other systems or devices). The controller 1414 and/or data acquisition unit 1426 may also be configured to exchange control signals with the platform 1490 (or components thereof), allowing the computing device 1410 to be used to control, for example, the position of the subject with respect to detection devices 1460 (e.g., in embodiments in which the platform 1490 is movable).

Data analyzer 1430 may direct analysis of the data and signals, and output analysis results. Data analyzer 1430 may be used, for example, to transform raw data captured via detection devices 1460, and may employ pre-processing procedures involved in generating a training dataset. For example, in some implementations, data may be generated as a multidimensional array or vector with values representing, and to prevent the machine learning system from overemphasizing certain readings, values may be normalized to a predetermined range (e.g. 0-1, 0-100, or any other such range). The normalization may comprise linear rescaling, or may be a more complex function. In some implementations, dimension reduction may be performed to reduce large and sparse arrays or vectors. In some implementations, feature recognition may be performed to select a subset of features for further analysis, such as principal component analysis.

A machine learning modeler 1440 may be used to implement various machine learning functionality discussed herein. Machine learning modeler 1440 may include a model training engine 1444 configured to train predictive models using, for example, data obtained from or via data acquisition unit 1426 and/or processed data obtained from or via data analyzer 1430. The model training unit 1444 may, for example, generate or obtain training datasets from or via data analyzer 1430 and may perform validation of datasets. The model training unit 1444 may comprise a feature analyzer used to evaluate features by, for example, quantifying the impact of each feature on the developed model. Such a feature analyzer may, for example, uncover clinically important features that were globally predictive of the outcome, and may determine, for example, contributions of all features, or the top features (e.g., the top 2, top 5, top 10, top 15, top 20, top 25, top 30, etc.) on individual predictions. Features may be selected based on a threshold, such a percent contribution to predicting a medical condition, such as 0.5%, 1%, 2%, 5%, 10%, etc. An application engine 1448 may be configured to apply models trained via model training engine 1444 to, for example patient data from data acquisition unit 1426 and/or data analyzer 1430.

A transceiver 1422 allows the computing device 1410 to exchange readings, control commands, and/or other data with detection devices 1460 (or components thereof). The transceiver 1422 may additionally or alternatively include a network interface permitting the computing device 1410 to communicate with other remote devices and systems via, for example, a telecommunications network such as the internet. One or more user interfaces 1418 allow the computing device 1410 to receive user inputs (e.g., via a keyboard, touchscreen, microphone, camera, etc.) and provide outputs (e.g., via display screen, audio speakers, etc.). A display screen may be employed, for example, to provide real time or near real time waveforms or other readings or measurements obtained via sensors being used to capture physiological data from subjects and patients. The computing device 1410 may additionally include one or more databases 1450 (stored in, e.g., one or more computer-readable non-volatile memory devices) for storing, for example, data and analyses obtained from or via data acquisition unit 1426, data analyzer 1430, machine learning modeler 1440 (e.g., model training engine 1444 and/or testing engine 1448), and/or detection devices 1460. In some implementations, database 1450 (or portions thereof) may alternatively or additionally be part of another computing device that is co-located or remote and in communication with computing device 1410 and/or detection devices 1460 (or components thereof).

### EXAMPLES

SpO₂ screening fails to detect many acyanotic defects with systemic obstruction such as coarctation of the aorta (CoA) and interrupted aortic arch (IAA). The majority of missed classifications are newborns with acyanotic systemic obstruction such as CoA and IAA. Unfortunately, late detection of these defects is particularly detrimental because the infants then present critically ill when surgical intervention may no longer prevent mortality or morbidity. It is estimated that increases in prenatal detection are unlikely to further enhance CCHD detection. Therefore, efforts aimed at improving postnatal detection of these life-threatening lesions are necessary. Unfortunately, other non-invasive methods, such as blood pressure gradient, overlap markedly between newborns with and without CCHD.

Peripheral perfusion index (PIx), a non-invasive measurement of pulsatile blood flow independent of oxygenation that can be measured simultaneously with SpO₂, can enhance the detection of CCHD, particularly defects such as CoA and IAA. However, the limited available literature does not provide consensus regarding normal PIx values and those indicative of CCHD. Additionally, current literature mostly includes PIx measurements in normal newborns and neonates with acyanotic systemic obstruction defects, and therefore it is not known if PIx may be abnormal in newborns with non-critical CHD or cyanotic defects.

In various embodiments, PIx enhances the detection of CCHD with systemic outflow obstruction among newborns that would otherwise not be detected by SpO₂ screening.

An example study corresponding to various potential embodiments included a single-center prospective cohort of newborns with and without CHD. The cohort of newborns without CHD was composed of asymptomatic newborns from the well newborn nursery. The only exclusion criterion for healthy newborns was parental refusal of CCHD screening. The cohort of newborns with CHD was derived of newborns with prenatally or postnatally identified CHD. The exclusion criteria for newborns with CHD were: (1) isolated patent ductus arteriosus and/or patent foramen ovale/atrial septal defect, (2) corrective surgical or catheter procedure prior to enrollment, and (3) active vasoactive infusions other than prostaglandin therapy.

Pre-ductal (right hand) and post-ductal (any foot) SpO₂ and PIx were measured in both cohorts. For the healthy cohort, PIx was measured after 24 hours of age, during the routine SpO₂ measurement for CCHD screening if it had not been completed prior to study enrollment. If routine CCHD screening had been completed prior to study enrollment, then a repeat SpO₂ and initial PIx were measured. Due to unpredictable circumstances for the CHD cohort, such as need for interventions, SpO₂ and PIx were not measured at specified times but were collected as soon as possible while noting presence or absence of prostaglandin therapy during the measurements. Study investigators collected the SpO₂ and PIx measurements for both cohorts. A single pre- and post-ductal SpO₂ and PIx value were recorded as soon as the waveform was artifact free for at least 10 seconds. Masimo Radical 7 pulse oximeters (Masimo Corp., Irvine, CA) were used for this study.

The electronic medical record (EMR) was reviewed for demographic and clinical characteristics. To confirm the healthy newborns were not classified with CHD at a later date, the EMR was reviewed for a well child physical after a minimum of 6 weeks of age. If a newborn did not have documented follow up within the EMR to confirm healthy status, parents were contacted by telephone to confirm their newborn was not later classified with a heart defect. If a newborn enrolled as a healthy newborn but was later found to have CHD, the newborn was analyzed as part of the CHD cohort and vice versa.

Classification of heart defects: Newborns with a defect requiring corrective surgical or catheter intervention in the first month after birth were classified as critical CHD (CCHD). Defects were also classified as either having systemic outflow obstruction or not. The result of a control newborn’s routine clinical SpO₂ screen was used when classifying as pass or fail. To classify a newborn with CHD as passing or failing SpO₂ screen, their routine SpO₂ screening result was used if available. If a newborn with CHD did not undergo routine SpO₂ screening before enrollment, then the SpO2 values measured during PIx measurements after 24 hours of life were used to classify as either passing or failing SpO₂ screen. SpO2 measurement was considered failing if (1) any SpO2 measurement was < 90%, (2) SpO₂ 90 to < 95% in both right hand and foot and/or a > 3% absolute difference between the right hand and foot on 3 measurements. Any SpO₂ measurement ≥ 95% in either the right hand or foot with ≤ 3% absolute difference was considered passing.

Pre- and post-ductal PIx and the absolute difference between pre- and post-ductal (PIx gradient) were recorded and compared between the cohorts. The 5^{th} percentile post-ductal PIx value among healthy controls was used retrospectively to classify newborns as failing PIx. Additional post-ductal PIx thresholds reported in the literature were also used to estimate sensitivity and specificity. Pre-ductal PIx was not used in the classification for failing or passing by PIx criterion. However, pre-ductal PIx has been used in other studies, therefore this example study evaluated the impact of pre-ductal PIx and the PIx gradient on sensitivity and specificity.

Summary statistics for the newborns with and without CHD were presented as medians or frequencies with interquartile ranges (IQRs) or percentages, respectively. The medians of continuous data were compared using the nonparametric equality-of-medians test. The Pearson chi square or Fisher exact test, as appropriate, was used to compare categorical data. Sensitivity and specificity were estimated with 95% Confidence Intervals (95% CI) and compared between screening strategies using McNemar’s Test. Ap value ≤ 0.05 was considered statistically significant. The data were analyzed with Stata Statistical Software, release 15.1 (Stata Corp, College Station, TX).

The final cohort of newborns without CHD was 123, which included 3 newborns with prenatally-suspected coarctation of the aorta all of whom were deemed to have normal cardiac anatomy by postnatal echocardiogram. Among the cohort of newborns without CHD, this example study had follow up data to confirm absence of CHD for 111 (90%) newborns by at least 6 weeks of age. The 12 (10%) newborns within this cohort that were lost to follow up however were included in the analysis. These 12 infants did not undergo any cardiac intervention at the two main cardiac programs in the region.

The final cohort of newborns with CHD was 21, of which 10 were suspected prenatally (Table 4). Thirteen (5 with systemic obstruction) of the 21 had CCHD. In addition to the 5 newborns with critical systemic obstruction defects, another newborn with aortic stenosis, mild CoA and mild hypoplastic mitral value that was not ductal dependent and did not require intervention in the neonatal period was enrolled for a total of 6 newborns with systemic outflow obstruction. One newborn initially enrolled as a healthy newborn was later found to have CHD (small ventricular septal defect) upon follow up, and this newborn was included in the CHD cohort. Additional details of the newborns with CHD are shown in Table 1. Demographic criteria did not differ between the newborns with and without CHD (Table 5).

Perfusion index: Pre- and post-ductal PIx did not differ between newborns with and without CHD (Table 6). Newborns with systemic outflow obstruction had a larger PIx gradient (absolute difference between the pre- and post-ductal values) compared to newborns without CHD, 0.9 (IQR 0.7-1.4) versus 0.4 (IQR 0.1-0.7) respectively (p = 0.01). The post-ductal PIx was lower in newborns with systemic obstruction compared to newborns without CHD, 0.5 (IQR 0.1-1.1) versus 0.9 (IQR 0.7-1.3) respectively, however this was not statistically significant (p = 0.68) (Table 6). All but one newborn with CCHD were receiving prostaglandin therapy during PIx measurements (Table 4).

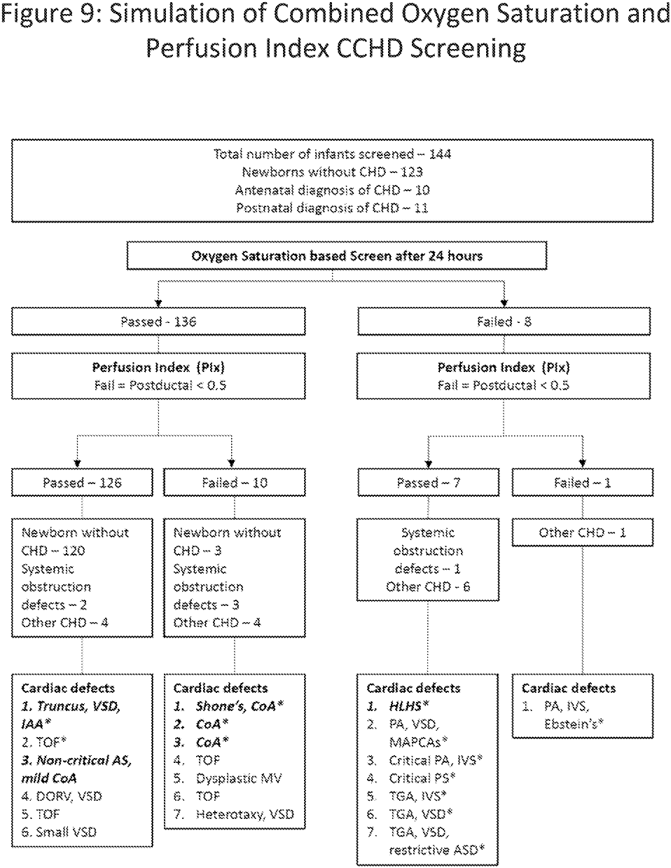

Oxygen saturation (SpO₂) screening: All infants (N=144, including 10 with antenatal suspicion and 11 with postnatal detection of CHD) underwent standard SpO₂-based CCHD screen (FIG. 9). One hundred and thirty six infants (including all 123 normal newborn infants without CHD) passed SpO₂-based CCHD screen. All the 11 newborns with postnatal detection of CHD (including 4 infants with critical systemic outflow obstruction) passed routine SpO₂ screening (if conducted prior to study enrollment) and were detected due to symptoms (Table 4 and FIG. 9). When applying the SpO₂ screening algorithm to the CHD cohort after enrollment, 8 failed by SpO₂ criterion (all had CCHD and included 1 with systemic obstruction and 7 without systemic obstruction) (FIG. 9). In the cohort, the sensitivity and specificity of SpO2-alone screening for CCHD versus healthy was 62% (95% CI 32-86%) and 100% (95% CI 97-100%) respectively (Table 7). For systemic obstruction CCHD versus healthy the sensitivity of SpO2-alone screening alone was 20% (95% CI 1-72%).

Oxygen saturation (SpO₂) + Perfusion index (PIx) screening: The 5^{th} percentile post-ductal PIx among newborns without CHD was 0.5. Based on these values, this example study defined a post-ductal PIx of < 0.5 as “failing” PIx values and applied these criteria to the cohort. Among the 136 infants that passed the SpO₂-based CCHD screen (FIG. 9), 10 infants failed the PIx-based screen based on criteria outlined above (postductal PIx < 0.5). This included 3 infants with critical systemic obstruction CCHD, 4 with other CHD and 3 normal infants. The sensitivity for CCHD versus healthy newborn of SpO₂-PIx combined screening was 85% (95% CI 55-99%) (Table 7), which compared to SpO2-alone was a difference of + 23 (95% CI - 8 to + 54, p = 0.08) (Table 7). For CCHD with systemic obstruction, the sensitivity of SpO₂-PIx combined screening was 80% (95% CI 28-100), which compared to SpO2-alone screening was as difference of + 60 (95% CI - 3 to 100, p = 0.08). Four of the 8 newborns with non-critical CHD, had failing PIx values. The sensitivity of SpO₂-PIx combined screening for all CHD versus healthy was 71% (95% CI 48-89%) (Table 7). Compared to SpO₂-alone the difference in sensitivity for SpO₂-PIx combined for CHD was + 33 (95% CI + 8 to 58, p = 0.008) (Table 7). The specificity of SpO₂-PIx combined screening (only considered healthy newborns as false positives) was 98% (95% CI 93-100%), which compared to SpO2-alone screening was a difference of - 2 (95% CI - 6 to + 1, p = 0.08) (Table 7).

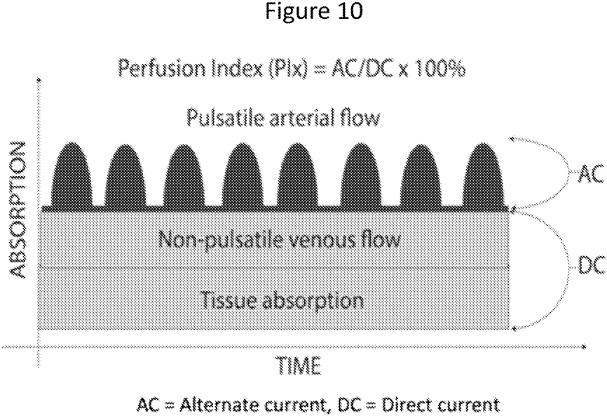

Newborns with systemic obstruction were more likely to have failing PIx values (50%) versus newborns without systemic obstruction (6%) (p = 0.006). Newborns with CHD, regardless of type, were more likely to have failing PIx values compared to newborns without CHD, 38% versus 2.44% respectively (p < 0.001). The three newborns with prenatally suspected coarctation of the aorta and postnatally determined to be normal all had “passing” PIx values (Table 4 and FIG. 10).

Other PIx threshold, pre-ductal PIx and PIx gradient as screening “failures”: A PIx value < 0.7 has been recommended as a threshold in prior studies.^{21,25} When using a post-ductal PIx < 0.7 in the cohort, in combination with SpO₂, as a screen failure criterion, the sensitivity and specificity for CCHD versus healthy were 85% (95% CI 55-98%) and 72% (95% CI 64-80%) respectively. When combining a pre- and post-ductal PIx < 0.5 with SpO2 as failure criterion, the sensitivity and specificity for CCHD vs healthy were 85% (95% CI 55-98) and 98% (95% CI 933-100%). When combining a PIx gradient (absolute difference between pre- and post-ductal) > 1.1 (the 95^{th} percentile for healthy newborns), SpO₂ and post-ductal PIx < 0.5, the sensitivity and specificity for CCHD vs healthy were 85% (95% CI 55-98%) and 93% (95% CI 87-97%) respectively.

CCHD lesions with systemic outflow tract obstruction, specifically CoA and IAA are commonly missed by SpO₂-based pulse oximetry screening. This example study demonstrated improved detection of these lesions with combined SpO2-PIx based screening. Sensitivity for CCHD improved from 62% to 85% with the addition of PIx to SpO₂ screening (p=0.08). This increase is likely from improved detection of defects with systemic obstruction. This is crucial because only one newborn with a critical systemic obstruction (hypoplastic left heart syndrome) defect in the cohort had failing SpO2 values and the remaining (80%, 3 CoA and one IAA) would have been missed by traditional SpO₂-based screening. Newborns with non-critical CHD may also be detected by PIx, as the sensitivity non-critical CHD improved from 38% to 71% with the addition of PIx to SpO₂. Screening with PIx however may result in a higher false positive rate, as 2.44% of the newborns without CHD had failing PIx results in the cohort.

This example study identified a lower PIx threshold for CCHD compared to prior studies. PIx values potentially indicative of CCHD vary in the literature, with three prior studies all identifying a different 5^{th} percentile values for a post-ductal PIx. Studies of thousands of healthy newborns even vary in the identified 5^{th} percentile PIx. Granelli at al. and Uygur et al. measured PIx with a similar approach as the example study, which involved documenting the PIx once the waveform was artifact free for approximately 10 seconds. However, they identified a 5^{th} percentile post-ductal PIx of 0.7 and 1.1 respectively. A third study that measured PIx as a convenience sample from SpO₂ screens, identified 0.6 as the 5^{th} percentile post-ductal PIx among normal newborns. The example study identified a lower potential threshold at 0.5, which may due to the smaller sample size in the example study. A PIx threshold of 0.5 however has been described as a “definite” state of under perfusion in larger studies. While the identified PIx threshold is the lowest among the literature, it is notable that a post-ductal PIx of 0.7 did not change the sensitivity for CCHD in the cohort. Additionally, the specificity for a post-ductal PIx of 0.7 decreased by an absolute difference of 26% (from 98% to 72%) compared to the identified PIx threshold of 0.5. Therefore, more studies are necessary to better identify a PIx threshold and estimate the impact on sensitivity and specificity.

The false positive rate will increase if PIx were added to the screening process and a better estimate of the false positive rate is needed. Two prior studies have estimated the false positive rate. Schena et al. conducted a prospective multicenter study including PIx in the screening process. Their false positive rate of the combined test was 0.45% (0.27% for PIx). However, one site in their study had a high false positive rate of 6%. Interestingly, both that outlier site and the hospital are tertiary hospitals, and Schena et al. noted a higher false positive among tertiary hospitals and suggested PIx may be more useful screening tool in non-tertiary hospitals. Uygur et al. estimated a false positive of 3.6% for a higher PIx post-ductal threshold of 1.1. Considering the false positive rate of SpO₂-based screening is extremely low (less than 0.2%), and that failing CCHD screening may require an echocardiogram or even transport to a facility with echocardiogram capabilities prior to discharge, an algorithm that optimizes both sensitivity and specificity is key. Additionally, it is notable that the lower PIx threshold of 0.5 used in the example study still resulted in a false positive rate over 10 times higher than that of SpO₂ alone.

The example was the only to date to analyze a difference between the pre- and post-ductal PIx. When the 95^{th} percentile of PIx gradient (absolute difference between pre- and post-ductal > 1.1) for normal newborns was included in the analysis as a failing PIx criterion, the false positive rate among normal newborns increased to 7%. Only 3 newborns with CHD, had a PIx gradient > 1.1 and all three of those newborns also had failing SpO₂ values. Therefore, PIx gradient (difference between pre-ductal and post-ductal measurement) may not be needed to enhance detection capability. Similarly, it may not be necessary to additionally use a pre-ductal PIx as part of the screening tool because the sensitivity and specificity did not differ when using both pre- and post-ductal PIx vs post-ductal PIx alone.

A strength of the example study is that the study included newborns with critical and non-critical CHD as well as newborns with and without systemic obstruction whereas prior studies with similar methods have only reported PIx values for newborns with critical systemic obstruction. Prior studies that have included other CHDs, have done so in a prospective screening of asymptomatic newborns and thus included smaller numbers of newborns with CHD. While CCHD screening is not primarily intended to detect non-critical defects, 4 of the 8 newborns with non-critical CHD had failing PIx values. All 4 of those newborns had passing SpO₂ values, and interestingly, 2 of them had Tetralogy of Fallot, the second most commonly missed classification by SpO₂ screening (second to CoA/IAA).¹⁵ Prior studies on SpO₂ screening demonstrated that SpO₂ could detect other important illnesses in newborns and non-critical CHD. The example study suggests PIx may perform similarly and have additional detection value.

Consequently, combining PIx with SpO₂ improves early detection of CCHD with systemic obstruction. Measurement of PIx values over several minutes enhances results due to the variation seen in PIx.

TABLE 4

Newborns with Suspected or Confirmed Congenital Heart Defects

| Heart defect | Pre/post ductal oxygen saturation | SpO₂ screen result | Pre/post ductal perfusion index | PIx screen result^{a} | Combined SpO₂ and PIx screen result |
|---|---|---|---|---|---|
| Critical congenital heart defects | | | | | |
| Systemic obstruction present | | | | | |
| Hypoplastic left heart syndrome^{e} | 86/85 | Fail | 1.6/3.1 | Pass | Fail |
| Shone’s complex, coarctation of aorta^{b,e} | 100/100 | Pass | 1.5/0.08 | Fail | Fail |
| Coarctation of aorta^{b,f} | 98/100 | Pass | 1.2/0.4 | Fail | Fail |
| Coarctation of aorta^{b,f} | 100/100 | Pass | 1.0/0.09 | Fail | Fail |
| Truncus, VSD, interrupted aortic arch^{b,e} | 94/96 | Pass | 0.68/1.1 | Pass | Pass |
| No systemic obstruction | | | | | |
| Pulmonary atresia, IVS, Ebstein’s anomaly^{b,e} | 84/80 | Fail | 0.53/0.28 | Fail | Fail |
| Transposition of great arteries, VSD^{b,f} | 75/80 | Fail | --^{c}/1.5 | Pass | Fail |
| Transposition of great arteries, VSD, ASD^{b,f} | 85/89 | Fail | 1.4/5.1 | Pass | Fail |
| Pulmonary atresia, VSD, MAPCAs^{b,e} | 77/93 | Fail | 0.41/1.1 | Pass | Fail |
| Critical pulmonary stenosis, IVS^{b,e} | 86/90 | Fail | 1.0/0.96 | Pass | Fail |
| Critical pulmonary stenosis^{b,f} | 87/83 | Fail | 1.3/1.1 | Pass | Fail |
| Transposition of great arteries, IVS^{b,e} | 70/70 | Fail | 1.2/1.5 | Pass | Fail |
| Tetralogy of Fallot^{b,f} | 98/97 | Pass | 2.4/1.6 | Pass | Pass |
| Non-critical congenital heart defects Systemic obstruction present | | | | | |
| AS, mild CoA, mild hypoplastic mitral valve^{f} | 100/100 | Pass | 1.3/0.65 | Pass | Pass |
| No systemic obstruction | | | | | |
| Tetralogy of Fallot^{e} | 99/99 | Pass | 0.91/0.25 | Fail | Fail |
| Dysplastic mitral valve and PHTN^{f} | 100/100 | Pass | 0.4/0.31 | Fail | Fail |
| Tetralogy of Fallot^{d,f} | 96/100, 99/100 | Pass | 1/0.48, 0.48/0.52 | Fail | Fail |
| Heterotaxy, VSD, PHTN^{e} | 98/96 | Pass | 1.1/0.4 | Fail | Fail |
| Double outlet right ventricle, VSD, no outlet obstruction^{d,e} | 90/91, 96/95 | Pass | 0.59/0.6, 1.4/0.64 | Pass | Pass |
| TOF^{f} | 96/94 | Pass | 1.9/2.6 | Pass | Pass |
| Small VSD - enrolled as control later classified^{f} | 100/100 | Pass | 1.7/1.1 | Pass | Pass |

“Coarctation of the aorta watches”

| | | | | | |
|---|---|---|---|---|---|
| Normal | 99/100 | Pass | 1.2/0.55 | Pass | Pass |
| Normal | 99/97 | Pass | 1.2/0.72 | Pass | Pass |
| Normal | 97/95 | Pass | 1.9/1.9 | Pass | Pass |

^{a} PIx result = fail if post-ductal perfusion index < 0.5.
^{b} Prostaglandin infusion during measurements.
^{c} Value would not register during measurement.
^{d} If SpO₂ values would trigger a repeat SpO₂ measurement, the repeat SpO₂ and PIx values are shown above.
^{e} First suspected prenatally.
^{f}First suspected postnatally.
Abbreviations: AS = aortic stenosis, CoA = coarctation of aorta, IVS = intact ventricular septum, MAPCAs = major aortopulmonary collateral arteries, PHTN = pulmonary hypertension, PIx = perfusion index, SpO₂ = oxygen saturation, VSD = ventricular septal defect

TABLE 5

Demographic Characteristic of Newborns with and without Congenital Heart Disease

| | All CHD (N = 21) | No CHD (N = 123) | P value |
|---|---|---|---|
| Age at first PIx measurement, hours, median (IQR) | 28 (23-53) | 36 (37-50) | 0.48 |
| Sex, male | 12 (57%) | 67 (54%) | 0.82 |
| Gestational age weeks, median (IQR) | 39 (36-40) | 39 (38-40) | 0.34 |
| Vaginal delivery | 12 (57%) | 64 (52%) | 0.67 |
| Race | | | 0.89 |
| American Indian/Alaskan | 0 | 2 (2%) | |
| Asian | 1 (5%) | 15 (12%) | |
| Native Hawaiian | 0 | 2 (2%) | |
| Black | 2 (10%) | 9 (7%) | |
| White | 14 (67%) | 67 (54%) | |
| More than one race | 0 | 1 (1%) | |
| Unknown/not reported | 4 (19%) | 27 (22%) | |
| Ethnicity | | | > 0.9 |
| Hispanic/Latino | 5 (24%) | 32 (26%) | |
| Not Hispanic/Latino | 16 (76%) | 87 (71%) | |
| Unknown/not reported | 0 | 4 (3) | |
| Family history of CHD present^{a} | 3 (16%) | 7 (6%) | 0.12 |

Abbreviations: CHD = congenital heart disease, PIx = perfusion index
^{a} Family history of CHD unknown for 2 of newborns with CHD and 2 without CHD

TABLE 6

Perfusion Index for Newborns with and without Congenital Heart Defects: Subgroup Analyses of Types of Heart Defect

| | | | | |
|---|---|---|---|---|
| | No CHD (N = 123) | All CHD (N= 21) | CHD Systemic obstruction (N = 6) | CHD No systemic obstruction (N = 15) |
| Pre-ductal PIx, median (IQR) | 1.3 (0.9-1.6) reference | 1.2 (0.6-1.6) p = 0.23 | 1.3 (1-1.5) p = 0.69 | 1.1 (0.6-1.4) p = 0.27 |
| Post-ductal PIx, median (IQR) | 0.9 (0.7-1.3) reference | 1 (0.4-1.5) p > 0.9 | 0.5 (0.1-1.1) p = 0.68 | 1.1 (0.4-1.5) p = 0.59 |
| PIx gradient^{a}, median (IQR) | 0.4 (0.1-0.6) reference | 0.7 (0.3-0.8) p = 0.06 | 0.9 (0.7-1.4) p = 0.01 | 0.6 (0.2-0.7) p = 0.59 |

Abbreviations: CHD = congenital heart disease, PIx = perfusion index
^{a} PIx gradient = absolute difference between pre- and post-ductal perfusion index
p-values are from nonparameteric equality-of-medians test, contrasting given group with the reference group of No-CHD infants.

TABLE 7

Sensitivity and Specificity of Oxygen Saturation and Perfusion Index Screening for Congenital Heart Defects

| | Oxygen Saturation Alone | Oxygen Saturation + Perfusion Index^{a} | p value |
|---|---|---|---|
| Critical Congenital Heart De fects^{b} (N = 13) | | | |
| Sensitivity, % (95% CI) | 62 (32-86) | 85 (55-98) | 0.08 |
| Sensitivity Difference, % (95% CI) | reference | 23 (- 8 to + 54) | NA |
| Critical Congenital Heart Def ects with Systemic O bstruction^{b} (N = 5) | | | |
| Sensitivity, % (95% CI) | 20 (1-72) | 80 (28-100) | 0.08 |
| Sensitivity Difference, % (95% CI) | reference | + 60 (-3 to 100) | NA |
| All Congenital Heart Defects^{b} (N = 21) | | | |
| Sensitivity, % (95% CI) | 38 (18-62) | 71 (48-89) | 0.008 |
| Sensitivity Difference, % (95% CI) | reference | + 33 (+ 8 to 58) | NA |
| No-CHD (N = 123) | | | |
| Specificity, % (95% CI) | 100 (97-100) | 98 (93-100) | 0.08 |
| Specificity Difference, % (95% CI) | Reference | - 2 (- 6 to +1) | NA |

^{a} Perfusion index result = fail if post-ductal perfusion index < 0.5. ^{b} For all comparison, McNemar’s Test was used to produce p-value and effect size and 95% CI were estimated using methods for paired binary data.

While SpO₂ screening has improved the early detection of CCHD, it is estimated that almost 900 US newborns with CCHD will be undetected annually despite prenatal ultrasound and SpO₂ screening. ^{15,} ²⁸ This in part due to misinterpretations of the SpO₂ screening algorithm by medical staff despite the algorithm’s simplicity.^{29,} ³⁰ Additionally, SpO₂ fails to detect many of the acyanotic targets (Table 5). The majority of missed classifications are acyanotic defects with obstructed blood flow to the body (systemic obstruction) such as coarctation of the aorta (CoA) or interrupted aortic arch (IAA).¹⁵ Unfortunately, late detection of these defects is particularly detrimental because the infants present critically ill, or even after a cardiac arrest, when surgical intervention may no longer prevent death or morbidity.¹⁷ The second most commonly missed CCHD is Tetralogy of Fallot (TOF), despite being traditionally a cyanotic lesion due to the potential of a “pink” (or acyanotic) TOF. CoA and TOF, the two defects with the lowest sensitivity for SpO₂ detection (Table 5), are also the two most common heart defects. As discussed above, the addition of non-invasive perfusion measurements may be used for detection of both of these defects as well as others.

TABLE 8

CCHD Screening Targets (Sensitivity of Oxygen Saturation Screening is shown as % in parentheses)
• Coarctation of the aorta or interrupted aortic arch (36%) - Most common defects with late detection
• Double-outlet right ventricle (57-100% - 95% Cl)*
• Ebstein’s anomaly (48%)
• Hypoplastic left heart syndrome (91%)
• Pulmonary atresia (44-100% - 95% Cl)*
• Tetralogy of Fallot (39%) - 2^{nd} most common defect with late detection
• Total anomalous pulmonary venous return (91%)
• Transposition of the great arteries (92%)
• Tricuspid atresia (44-97% - 95% Cl)*
• Truncus arteriosus (91%)
• Single ventricle (71%)
*For less frequent lesions, authors provided 95% confidence interval for sensitivity.

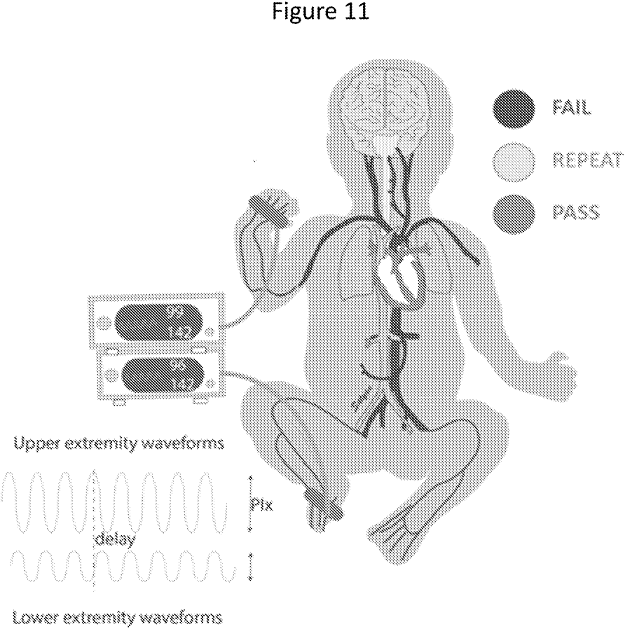

Perfusion index (PIx) is a non-invasive measurement of pulsatile blood flow independent of oxygenation that can be measured simultaneously with devices currently used to measure SpO₂ (FIG. 10). As discussed above, the addition of PIx to SpO2 screening improves sensitivity from 20% to 80% for newborns with systemic obstruction CCHD such as CoA/IAA. PIx was also noted to be abnormal in the two infants with acyanotic TOF in that study. However, the PIx also varies over brief periods of time (seconds). The PIx variation is in part due to its sensitivity to factors that affect vascular tone (i.e. sympathetic nervous system tone from a crying baby). PIx is also an unfamiliar measurement for clinicians. Therefore, incorporation of PIx in its current form into the CCHD screening algorithm is anticipated to be fraught with errors and misinterpretations - worse so than the misinterpretations already encountered in the SpO₂ screen. Therefore, various embodiments combine multiple measurements of SpO2 and PIx (e.g., over minutes), filter out false values associated with artifact, and classify a baby’s CCHD screen as, for example, “pass” or “fail” (FIG. 11). This approach makes practical use of PIx measurements in combination with SpO2 to save newborn lives.

In various embodiments, PIx and other pulse oximetry characteristics may be used in detecting CCHD. Various embodiments simultaneously measure pre and post ductal pulse oximetry for a time period, such as 5-minutes, collecting non-invasive measurements of oxygenation and perfusion. Machine learning techniques, such as supervised machine learning and k-fold validation, are used to develop and test a predictive model that combines measures of oxygenation and perfusion. Instead of using a subjective, spot check of oxygen saturation, this approach aggregates a time period of simultaneous pre-ductal and post-ductal SpO2 and PIx to objectively determine whether the infant has passed, failed, or needs a repeat test.

Various embodiments may utilize a PIx threshold of, for example, less than 0.5 or 0.7 to prompt further investigation for CCHD. User of such thresholds, however, may be only brief snapshots in time (10 seconds) and do not take into consideration large variation in PIx over brief time periods. Various embodiments may, for example, average PIx and only incorporate “validated” values into the predictive model.

Various embodiments enable simultaneous data collection from two pulse oximeters. Example embodiments code Pi-Top laps to simultaneous collect and display pulse oximetry data from two Nonin pulse oximeters. The Pi-Top/Nonin configuration may incorporate automated interpretation. The data collected on the Pi-Top/Nonin may be analyzed through application of machine learning techniques to identify characteristics associated with CCHD. Characteristics include averaged, minimum, maximum, median, and variance of SpO2 and PIx. Various embodiments recognize that values associated with motion artifact should not be incorporated into those averages, minimums, maximums, medians, or variances. In various embodiments, measurements using Nonin pulse oximeters and filtering out of artifacts provide an averaged, minimum, maximum, median and variance for SpO2 and PIx after incorporating values only associated with motion artifact free measurements. In various embodiments, readings of “pass” or “fail” may be based on Kemper SpO2 CCHD screening thresholds, in addition to a threshold for PIx, and optionally components related to radiofemoral delay, PPG slope, PPG image, and/or heart rate as well.

There are nearly 4 million babies born in the US annually. All US newborns, and most internationally, undergo SpO2 based CCHD screening with pulse oximeters. Currently, a nurse places a pulse oximeter probe on the right hand to spot check SpO2 and repeats the procedure in a foot and records these numbers with manual interpretation - a process that is time consuming and prone to errors. This current process also does not factor in markers of perfusion, which may be used to improve CCHD detection. Various embodiments comprise a device programmed for simultaneous pre and post ductal pulse oximetry measurement and automated interpretation of SpO2 and PIx.

Various embodiments improve detection of acyanotic defects such as CoA and IAA by adding PIx and pulse-oximetry waveform analysis to the current screening process. In various embodiments, PIx is a non-invasive measurement of pulsatile flow that can be measured with the current equipment used for SpO2 screening. If absolute PIx value and the pulse delay from the upper to lower extremity, or radiofemoral delay, are abnormal in a newborn, there may be defects such as CoA and IAA.

Despite its non-invasive mode and potential clinical application, PIx may suffer from variability over brief time periods (seconds). Various embodiments incorporate PIx into a predictive model involving multiple measurements.

Various embodiments enable enhanced CCHD detection by combining such parameters as oxygenation and perfusion in a predictive model. Upper and lower extremity SpO2 and PIx may be measured, and a PIx threshold for CCHD may be selected. In some embodiments, a lower extremity PIx of, for example, 0.5 may be identified as a threshold to trigger evaluation for CCHD. CCHD screening models that combine non-invasive measurements of perfusion, oxygenation (SpO2-PIx) and waveform characteristics may be employed. Supervised machine learning with cross-validation may be used to train an SpO2-PIx model that will categorize a newborn’s screen into “pass,” “fail,” or “requires repeat testing.” The machine-learning trained predictive model may improve sensitivity of CCHD detection with little impact on specificity compared to SpO2-alone.

In various embodiments, PIx, a non-invasive measurement of pulsatile blood flow that is independent of oxygenation and that can be measured simultaneously with devices used to measure SpO2. PIx may be determined by expressing the pulsatile, or alternate current (AC), of the photoplethysmogram as a percentage of the non-pulsatile, or direct current (DC) made up of absorbed light by the remaining tissue and venous flow (FIG. 10). The addition of PIx may improve the detection of CCHD missed by SpO2 screening. PIx has been shown to correlate well with cardiac output. PIx is also a marker of non-cardiac critical illnesses in newborns (chorioamnionitis, sepsis, pneumonia, bronchopulmonary dysplasia, intraventricular hemorrhage and necrotizing enterocolitis). Despite its potential clinical utility and non-invasive measurement, PIx is underused today. This is likely due to the variability in PIx measurements over brief time periods (seconds). PIx is sensitive to factors that affect vascular tone such as temperature, vasoactive drugs, sympathetic nervous system tone (i.e. pain, a crying baby, etc.) and stroke volume. Consequently, multiple PIx measurements over time (e.g., over one or more minutes, such as two minutes, three minutes, five minutes, etc.) may be clinically more useful to a predictive model for detecting CCHD.

In certain embodiments, a single PIx value may be documented after a waveform is artifact free for a certain amount of time, such as 5 or 10 seconds. When measured this way, the variability of PIx over brief time periods (seconds) likely contributes to variations in sensitivity estimates. Additionally, a lack of familiarity with PIx may result in inaccurate interpretations. In various embodiments, a predictive model may employ multiple PIx measurements automates interpretation and offsets these limitations. PIx may enhance detection value in specific types of clinical settings such as resource limited settings.

In various embodiments, in addition to the absolute PIx value, the delay in the pulse from the upper to lower extremity (radiofemoral delay) may enhance the detection of CoA and IAA. Pulse delay has been shown to be impacted by patency/closure of the ductus arteriosus and to correlate with stroke volume. CCHD screening is intended to identify newborns much younger (within a few days after birth) and when the ductus arteriosus is open. Therefore, radiofemoral delay in younger newborns may play an important role in the predictive model. Additionally, interpretation of radiofemoral delay is too complicated for bedside use.

Various embodiments will improve detection of critical congenital heart disease reducing mortality and morbidity associated with late classification. This has the potential to vastly improve sensitivity of CCHD screening for acyanotic defects, and ease the interpretation of the screening results with automatic data processing.

In various embodiments, PIx improve CCHD detection through inclusion of multiple PIx measurements, which will offset the variations of PIx over brief time periods (seconds) and therefore improve its clinical utility. A predictive model combines noninvasive measurements of oxygenation and perfusion to categorize a newborn’s SpO2-PIx measurements as, for example, “pass,” “fail,” or “requires repeat testing,” easing the screening process and interpretation.

In a study, the sensitivity for critical systemic obstruction improved from 20% (95% CI 1-72) to 80% (95% CI 28-100), with a difference of +60% (p = 0.08). Among the newborns with passing SpO2 results, newborns with systemic obstruction were more likely to have failing PIx values (60%) versus newborns without systemic obstruction (10%) (p = 0.01). Additionally, among the 8 newborns with non-critical CHD, 4 had failing PIx values. Notably, 2 of these 4 newborns had Tetralogy of Fallot, the second most commonly missed classifications by SpO2-based screening (Table 6). Thus, similarly to SpO2 screening, PIx screening may detect additional diseases/defects.

Various embodiments measure PIx once the signal is artifact free for a certain minimum time, such as at least 3 seconds, at least 5 seconds, at least 10 seconds, at least 30 seconds, etc. The variation of PIx over brief time periods (seconds) may result in values that truly reflect the physiology. Thus, various embodiments measure the PIx over a longer period (e.g., one or more minutes, such as 2, minutes, 3 minutes, 5 minutes, 10 minutes or longer).

The predictive model may output a result based on input parameters. Various embodiments may incorporate SpO2, PIx, and waveform analysis simultaneously on upper and lower extremities in newborns. In various embodiments, a lower extremity PIx (of, e.g., 0.5) may be identified as a threshold to trigger evaluation for CCHD. Cross-validation may be used to test models that combine non-invasive measurements of oxygenation and perfusion. Various embodiments may use machine learning techniques, such as supervised learning.

To acquire training data for use in various embodiments, data from a two-year prospective cohort study of newborns with and without CHD may be implemented. Inclusion criteria may be: 1) age < 7 days and either 2a) asymptomatic newborn undergoing SpO2 screening for CCHD, or 2b) newborn prenatally or postnatally classified with CHD. Exclusion criteria for newborns with CHD may be: 1) patent ductus arteriosus and/or atrial septal defect/patent foramen ovale without other defects, 2) corrective surgical or catheter intervention performed before enrollment, and 3) current infusions of vasoactive medications other than prostaglandin therapy.

With respect to chart review and data collection, demographic and clinical characteristics may be extracted from the electronic medical record (EMR) for disease classification. EMR data need not be used in the machine learning model. To confirm healthy controls were not later classified with CHD, EMRs will be reviewed up to 6 months of age for well child visits confirming or disproving healthy status. If the child does not follow up within a study site, the parents or pediatrician will be contacted to confirm the child was not classified later with CHD. PIx, SpO2, and waveform analysis will be measured in all participants over 5 minutes using motion-tolerant pulse oximeters (Masimo Radical 7 and Nonin WristOx2 Model 3150 OEM). The strengths of the two devices will be combined: 1) Bedside clinician interpretation of PIx (Masimo) and 2) High resolution photoplethysmography waveform to decipher artifact and continuous Bluetooth data (Nonin). Pre-ductal (right hand) and post-ductal (any foot) sites will be measured simultaneously. Researchers will document SpO2 and PIx when the signal is artifact free for 10 seconds to be used in analysis for AIM 1. The photoplethysmography waveforms, PIx, SpO2, heart rate, and signal quality will be downloaded directly to small computer devices, Raspberry-Pis, to be used for machine learning models.

In various embodiments, as a preliminary step to model development, a variety of SpO2 and PIx parameters will be described, including means and key quantiles (e.g. 1st, 5th, 25th, 50th, 75th, 95th, and 99th percentiles). The distributions of SpO2 and PIx may be summarized by estimated quantiles and respective 95% confidence intervals (CI). The distributions of the non-invasive perfusion and oxygenation variables may be compared between newborns with and without CHD graphically and using nonparametric area under the receiver operating characteristic curve (AUROC) analyses. Newborns with CHD may also be classified as non-critical CHD vs critical CHD (CCHD), and presence or absence of systemic obstruction. The non-invasive perfusion and oxygenation variables may be compared among the different types of CHD.

In various embodiments, SpO2, PIx, radiofemoral delay, and photoplethysmography waveforms from the cohort of newborns with and without CHD may be analyzed with a variety of machine learning (ML) techniques to train a classifier model that combines non-invasive measurements of oxygenation, perfusion and waveform characteristics. Various embodiments may utilize clinicians to create expert knowledge-derived heuristic methodology. Signal artifact caused by subject movement and delivery of care can result in inaccurate PIx and SpO2 values and is a danger to model performance. Therefore, values associated with signal artifact may be eliminated from analysis. Various embodiments may develop and employ several clinically relevant rules to filter the majority of signal artifact, using signal processing techniques such as wavelet transforms and dynamic time warping to filter the residual artifact. Various embodiments employ ML techniques to train a superior disease classifier compared to clinician derived rulesets.

Machine Learning Based Approach: Feature Extraction/Selection and Classification Methodology: An important step in developing various embodiments of a predictive model is feature selection methods to reduce the dimensionality of the dataset, speed model training time, and improve model performance.³² Feature selection can be performed through expert knowledge or using computational methods. Performance of prediction models may be compared using both features defined by expert clinicians as well as computation methods, including the Chi-square test, Recursive Feature Elimination, Principal Component Analysis, Linear Discriminant Analysis, and Independent Component Analysis.

In various embodiments, the disclosed approach may involve training a predictive model employing one or more machine learning techniques for detecting heart defects. For example, a classifier or other predictive machine learning model may be trained (e.g., via supervised, semi-supervised, or unsupervised learning) using data measurements from subjects with known heart defects, and the trained model applied to data on measurements from patients not known to have defects. One or more suitable machine learning techniques may be used alone or in combination in training and applying models.

Various embodiments may utilize different classifiers or combinations thereof: Naive Bayes Classifier, K-Nearest Neighbors, Decision Tree, Support Vector Machine, Gradient Boosting Classifier, Random Forest (RF) (FIG. 12), and/or Logistic Regression (LR) For model training and testing, various embodiments may use cross patient learning to segregate specific patients into a training cohort, and others into a testing cohort. To protect against overfitting bias, various embodiments may perform k-fold validation (i.e., creating k partitions of the data, randomly select one for testing and using the remaining k-1 partitions for training). For model development, various embodiments may employ sensitivity and specificity as primary metrics. In training and validation data sets, the relatively low proportion of newborns with systemic obstruction such as CoA may result in a class imbalance problem, leading to inaccurate classification. Various embodiments may employ synthetic minority over-sampling technique (SMOTE) to overcome class imbalance problems.³³ Optimal mixed methods for feature selection (which may include features from both statistical methods and expert knowledge) may be employed in various embodiments, and potentially ensemble methods (a combination of classifiers) to optimize the performance of the model.

Comparison of newly developed algorithms to SpO2-alone screening: Following the development of the algorithms that will classify a newborn’s screen as “fail,” “pass,” or “requires repeat testing,” various embodiments may employ techniques for paired receiver operating characteristic curves to compare the discriminative capacity of the SpO2-PIx clinician interpreted algorithm and the machine learning automated algorithm to the current SpO2-alone screen. The 95% CI may be calculated for sensitivity and specificity for each algorithm. Various embodiments may employ McNemar’s Test and 95% CI for differences in paired proportions to compare classification performance for compared algorithms, separately for cases (to compare sensitivity) and for controls (to compare specificity). Model development may incorporate perfusion measurements that improve sensitivity of CCHD detection with little impact on specificity compared to SpO2-alone.

With respect to sample size justification, enrollment of 200 healthy newborns and at least 20 newborns with acyanotic CCHD (CoA and IAA) is expected to ensure sufficient power to detect when the prediction model that combines PIx and SpO2 provides significant improvement in discriminative capacity for acyanotic CCHD. SpO2-based screening has a near-perfect specificity but sensitivity of approximately 36% for acyanotic CCHD, a value that corresponds to an AUROC of 68% (using the algebraic relationship that the AUROC for a binary test is 0.5*(Sensitivity + Specificity)). Example embodiments tested the null hypothesis that the AUROC for the SpO2-PIx algorithm is the same as the rule that uses SpO2-alone, but example embodiments considered a clinically significant improvement in discriminative capacity to obtain an AUROC of 85%, which corresponds to improving sensitivity to 73% for a cut-off that achieves 97% specificity. According to the SAS ROCPOWER macro, the planned sample size will provide 81% power to detect the clinically significant improvement (an AUROC of 85%) when performing a 2-sided test (with alpha=5%) of the null hypothesis of no difference, under the assumption that the AUROC for the SpO2-only rule is 68%.38

Some newborns with CCHD will be receiving prostaglandin therapy to maintain patency of the ductus arteriosus per standard treatment when enrolled. The patency of the ductus may impact the PIx measurement, however the goal of CCHD screening is to identify newborns with CCHD when that ductus is still open. Therefore, demonstrating abnormal PIx in the presence of prostaglandin therapy would further support adding it to CCHD screening. Additionally, results have demonstrated that three-quarters of newborns with systemic obstruction had abnormally low PIx while receiving prostaglandin therapy. It is expected that PIx values will be even lower or more likely to be in the “failing” range in the absence of prostaglandin therapy.

Some newborns with CCHD will undergo corrective intervention within a few days after birth or require vasoactive medications making them ineligible for enrollment, thus leaving a small window for enrollment. To offset this, researchers will receive alerts from the high-risk delivery team in the neonatal intensive care unit and daily pharmacy alerts for prostaglandin therapy orders, which were effective in a prior study. Women pregnant with a fetus suspected to have CHD can be consented prior to giving birth, which was effective in the prior study. Researchers will have HIPAA waivered access to the EMR list of women pregnant with fetuses suspected to have CCHD to screen for eligibility.

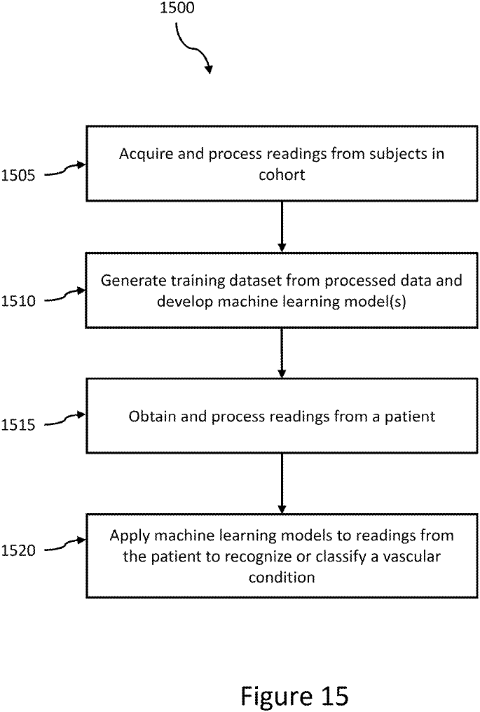

A flowchart for an example process 1500 according to various potential embodiments is shown in FIG. 15. At 1505, physiological readings are acquired from subjects in a cohort (e.g., via detection devices 1460) and may be analyzed or otherwise processed (e.g., by data analyzer 1430). The cohort may include subjects with a vascular condition as well as control subjects without the vascular condition. In certain embodiments, the controller 1414 may, for example, instruct detection devices 1460 to acquire and provide readings to computing device 1410. Raw test results may be processed (e.g., by or data analyzer 1430). At 1510, a training dataset may be generated from readings and one or more machine learning models may be developed as disclosed herein (e.g., by or via machine learning modeler 1440). At 1515, physiological readings from a patient (who may or may not have the medical condition) may be acquired via detection devices 1460. For example, the controller 1414 may instruct detection devices 1460 to acquire readings and provide readings to computing device 1410. The trained models may be applied (by, e.g., testing engine 1448) to the readings from tests on the patient’s to determine whether the patient has the vascular condition or to determine a severity of the vascular condition.

This approach has the potential to save hundreds of newborns lives in the US annually. Additionally, it will change the current standard screening method for CCHD by using non-invasive measurements that are not currently used in this manner. This approach may measure non-invasive perfusion measurements in the largest cohort of newborns with CHD and the target defects (CoA/IAA) and lead to automated CCHD predictive models combining non-invasive measurements of perfusion and oxygenation. The multicenter approach will allow for efficient enrollment of newborns with the target CCHD and establish the necessary infrastructure for large multicenter studies for later stages.

In various embodiments, pulse oximetry components that may be selected for inclusion in predictive modeling include: 1) SpO2 oxygen saturation; 2. Perfusion index or pulse amplitude index (synonymous measurements); 3. Radiofemoral delay based on simultaneous hand and foot measurements; 4. Photoplethysmography waveform slopes; 5. Heart rate data, comprising rate and/or variability or lack of variability (which may be measured, e.g., without using pulse oximetry for enhanced fidelity); and/or 6. Image of the photoplethysmography waveforms.

Various embodiments employ simultaneous hand and foot measurements, however only the radiofemoral delay component may require the simultaneous component. Because not all pulse oximeters allow for simultaneous measurements, various embodiments may allow for either sequential or simultaneous measurements.

### Definitions

As it would be understood, the section or subsection headings as used herein is for organizational purposes only and are not to be construed as limiting and/or separating the subject matter described.

Unless defined otherwise, all technical and scientific terms used herein have the same meanings as commonly understood by one of ordinary skill in the art to which this disclosure belongs. Although any methods and materials similar or equivalent to those described herein can be used in the practice or testing of the present disclosure, the preferred methods, devices, and materials are now described. All technical and patent publications cited herein are incorporated herein by reference in their entirety. Nothing herein is to be construed as an admission that the disclosure is not entitled to antedate such disclosure by virtue of prior disclosure.

As used in the specification and claims, the singular form “a,” “an” and “the” include plural references unless the context clearly dictates otherwise. For example, the term “a cell” includes a plurality of cells, including mixtures thereof.

As used herein, the term “comprising” is intended to mean that the compounds, compositions and methods include the recited elements, but not exclude others. “Consisting essentially of” when used to define compounds, compositions and methods, shall mean excluding other elements of any essential significance to the combination. Thus, a composition consisting essentially of the elements as defined herein would not exclude trace contaminants, e.g., from the isolation and purification method and pharmaceutically acceptable carriers, preservatives, and the like. “Consisting of” shall mean excluding more than trace elements of other ingredients. Embodiments defined by each of these transition terms are within the scope of this technology.

As will be understood by one skilled in the art, for any and all purposes, all ranges disclosed herein also encompass any and all possible subranges and combinations of subranges thereof. Furthermore, as will be understood by one skilled in the art, a range includes each individual member.

It is noted that terms such as “approximately,” “substantially,” “about,” or the like may be construed, in various embodiments, to allow for insubstantial or otherwise acceptable deviations from specific values. In various embodiments, deviations of 20 percent may be considered insubstantial deviations, while in certain embodiments, deviations of 15 percent may be considered insubstantial deviations, and in other embodiments, deviations of 10 percent may be considered insubstantial deviations, and in some embodiments, deviations of 5 percent may be considered insubstantial deviations. In various embodiments, deviations may be acceptable when they achieve the intended results or advantages, or are otherwise consistent with the spirit or nature of the embodiments.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

The term “subject,” “host,” “individual,” and “patient” are as used interchangeably herein to refer to animals, typically mammalian animals. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject is suspected of having a medical condition. In further embodiments, the subject may be asymptomatic. In other embodiments, the subject may be symptomatic, i.e., showing a symptom of the medical condition.

A decision tree is a tree-like model of decisions and their possible consequences, including chance event outcomes, resource costs, and utility, displaying an algorithm that only contains conditional control statements. Ensemble methods combine several decision trees to produce better predictive performance than utilizing a single decision tree. Ensembled decision trees may be bagged or boosted.

Bagging (Bootstrap Aggregation) is used to reduce the variance of a decision tree, for example by creating several subsets of data from training sample chosen randomly with replacement, using each collection of subset data to train the decision trees, and accordingly ending up with an ensemble of different models. Average of all the predictions from different trees are used which is more robust than a single decision tree. One non-limiting example of bagged decision trees is random forest, which takes one extra step using the radom selection of features rather than using all features to grow trees.

Boosting is another ensemble technique to create a collection of predictors. In this technique, learners are learned sequentially with early learners fitting simple models to the data and then analyzing data for errors. Consecutive trees (random sample) are fitted and at every step, the goal is to solve for net error from the prior tree. When an input is misclassified by a hypothesis, its weight is increased so that next hypothesis is more likely to classify it correctly. By combining the whole set at the end converts weak learners into better performing model. Gradient Boosting is an extension over boosting method, using gradient descent algorithm which can optimize any differentiable loss function. An ensemble of trees are built one by one and individual trees are summed sequentially. Next tree tries to recover the loss (difference between actual and predicted values). Also, see wikipedia.org/wiki/Gradient_boosting for more details about gradient boosting which is enclosed herein by reference in its entirety. Non-limiting examples of gradient boosting include Light Gradient Boosting Machine(LightGBM), XGBoost, or Adaptive Boosting (AdaBoost).

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

It should be understood that although the present invention has been specifically disclosed by certain aspects, embodiments, and optional features, modification, improvement and variation of such aspects, embodiments, and optional features can be resorted to by those skilled in the art, and that such modifications, improvements and variations are considered to be within the scope of this disclosure.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the claims. Such variations will depend on the machine-readable media and hardware systems chosen and on designer choice. It is understood that all such variations are within the scope of the disclosure. Likewise, software and web implementations of the present disclosure may be accomplished with standard programming techniques with rule based logic and other logic to accomplish the various database searching steps, correlation steps, comparison steps and decision steps.

The embodiments described herein have been described with reference to drawings. The drawings illustrate certain details of specific embodiments that provide the systems, methods and programs described herein. However, describing the embodiments with drawings should not be construed as imposing on the disclosure any limitations that may be present in the drawings.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the claims. Such variations will depend on the machine-readable media and hardware systems chosen and on designer choice. It is understood that all such variations are within the scope of the disclosure. Likewise, software and web implementations of the present disclosure may be accomplished with standard programming techniques with rule based logic and other logic to accomplish the various database searching steps, correlation steps, comparison steps and decision steps.

In addition, where features or aspects of the present technology are described in terms of Markush groups, those skilled in the art will recognize that the present technology is also thereby described in terms of any individual member or subgroup of members of the Markush group.

It should be noted that the terms “exemplary,” “example,” “potential,” and variations thereof, as used herein to describe various embodiments, are intended to indicate that such embodiments are possible examples, representations, or illustrations of possible embodiments (and such terms are not intended to connote that such embodiments are necessarily extraordinary or superlative examples).

The term “coupled” and variations thereof, as used herein, means the joining of two members directly or indirectly to one another. Such joining may be stationary (e.g., permanent or fixed) or moveable (e.g., removable or releasable). Such joining may be achieved with the two members coupled directly to each other, with the two members coupled to each other using a separate intervening member and any additional intermediate members coupled with one another, or with the two members coupled to each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

The term “or,” as used herein, is used in its inclusive sense (and not in its exclusive sense) so that when used to connect a list of elements, the term “or” means one, some, or all of the elements in the list. Conjunctive language such as the phrase “at least one of X, Y, and Z,” unless specifically stated otherwise, is understood to convey that an element may be either X, Y, Z; X and Y; X and Z; Y and Z; or X, Y, and Z (i.e., any combination of X, Y, and Z). Thus, such conjunctive language is not generally intended to imply that certain embodiments require at least one of X, at least one of Y, and at least one of Z to each be present, unless otherwise indicated.

References herein to the positions of elements (e.g., “top,” “bottom,” “above,” “below”) are merely used to describe the orientation of various elements in the Figures. It should be noted that the orientation of various elements may differ according to other exemplary embodiments, and that such variations are intended to be encompassed by the present disclosure.

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

It is important to note that the construction and arrangement of the devices, assemblies, and steps as shown in the various exemplary embodiments is illustrative only. Additionally, any element disclosed in one embodiment may be incorporated or utilized with any other embodiment disclosed herein. Although only one example of an element from one embodiment that can be incorporated or utilized in another embodiment has been described above, it should be appreciated that other elements of the various embodiments may be incorporated or utilized with any of the other embodiments disclosed herein.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

All publications, patent applications, patents, and other references mentioned herein are expressly incorporated by reference in their entirety, to the same extent as if each were incorporated by reference individually. In case of conflict, the present specification, including definitions, will control.

Additional background and supporting information can be found in the following documents, each of which is incorporated herein by reference in its entirety:

1. Reller MD, Strickland MJ, Riehle-Colarusso T, Mahle WT, Correa A. Prevalence of Congenital Heart Defects in Metropolitan Atlanta, 1998-2005. JPediatr. 2008;153(6):807-813. doi:10.1016/j.jpeds.2008.05.059.

2. Matthew E Oster, Kyung a Lee, Margaret a Honein, Tiffany Riehle-Colarusso, Mikyong Shin AC. Temporal Trends in Survival Among Infants with Critical Congenital Heart Defects. Pediatrics. 2013;131(5):e1502. doi:10.1542/peds.2012-3435.Temporal.

3. Wren C, Reinhardt Z, Khawaja K. Twenty-year Trends in Diagnosis of Life-threatening Neonatal Cardiovascular Malformations. Arch Dis Child Fetal Neonatal Ed. 2008;93(1):F33-F35. doi:10.1136/adc.2007.119032.

4. Koppel RI, Druschel CM, Carter T, et al. Effectiveness of Pulse Oximetry Screening for Congenital Heart Disease in Asymptomatic Newborns. Pediatrics. 2003;111:451-455. doi:10.1542/peds.111.3.451.

5. Abu-Harb M, Hey E, Wren C. Death in Infancy from Unrecognised Congenital Heart Disease. Arch Dis Child. 1994;71(1):3-7. doi:10.1136/adc.71.1.3.

6. Gilboa S, Salemi J, Nembhard W, Fixler D, Correa A. Mortality Resulting from Congenital Heart Diseae Among Children and Adults in the United States, 1999 to 2006. Circulation. 2010;122(22):2254-2263. doi:10.1161/CIRCULATIONAHA.110.947002.Mortality.

7. Hunter S, Heads A, Wyllie J, Robson S. Prenatal Diagnosis of Congenital Heart Disease in the Northern Region of England: Benefits of a Training Programme for Obstetric Ultrasonographers. Heart. 2000;84(3):294-298. doi:10.1136/heart.84.3.294.

8. Randall P, Brealey S, Hahn S, Khan KS, Parsons JM. Accuracy of Fetal Echocardiography in the Routine Detection of Congenital Heart Disease Among Unselected and Low Risk Populations: A Systematic Review. BJOG. 2005;112(1):24-30. doi:10.1111/j.1471-0528.2004.00295.x.

9. Westin M, Saltvedt S, Bergman G, et al. Routine Ultrasound Examination at 12 or 18 Gestational Weeks for Prenatal Detection of Major Congenital Heart Malformations? A Randomised Controlled Trial Comprising 36 299 Fetuses. BJOG. 2006;113(6):675-682. doi:10.1111/j.1471-0528.2006.00951.x.

10. Quartermain MD, Pasquali SK, Hill KD, Goldberg DJ, Huhta JC. Variation in Prenatal Diagnosis of Congenital Heart Disease in Infants. Pediatrics. 2015;136(2). doi:10.1542/peds.2014-3783.

11. Manja V, Mathew B, Carrion V, Lakshminrusimha S. Critical Congenital Heart Disease Screening by Pulse Oximetry in a Neonatal Intensive Care Unit. J Perinatol. 2015;35(1):67-71. doi:10.1038/jp.2014.135.

12. Mahle WT, Newburger JW, Matherne GP, et al. Role of Pulse Oximetry in Examining Newborns for Congenital Heart Disease: A Scientific Statement from the AHA and AAP. Pediatrics. 2009;124(2):823-836. doi:10.1542/peds.2009-1397.

13. de-Wahl Granelli A, Wennergren M, Sandberg K, et al. Impact of Pulse Oximetry Screening on the Detection of Duct Dependent Congenital Heart Disease: A Swedish Prospective Screening Study in 39 821 Newborns. BMJ. 2008;337:a3037. doi:10.1136/bmj.a3037.

14. Secretary of Health & Human Services Letter to the Secretary’s Advisory Committee on Heritable Disorders in Newborns and Children (SACHDNC); Sep. 21, 2011.

15. Ailes EC, Gilboa SM, Honein MA, Oster ME. Estimated Number of Infants Detected and Missed by Critical Congenital Heart Defect Screening. 2015;135(6). doi:10.1542/peds.2014-3662.

16. Abouk R, Grosse S, Ailes E, Oster M. Association of US State Implementation of Newborn Screening Policies for Critical Congenital Heart Disease With Early Infant Cardiac Deaths. JAMA. 2017;318(21):2111-2118. doi:10.1001/jama.2017.17627.

17. Lannering K, Bartos M, Mellander M. Late Diagnosis of Coarctation Despite Prenatal Ultrasound and Postnatal Pulse Oximetry. Pediatrics. 2015;136(2). doi:10.1542/peds.2015-1155.

18. Patankar N, Fernandes N, Kumar K, Manja V, Lakshminrusimha S. Does Measurement of Four-limb Blood Pressures at Birth Improve Detection of Aortic Arch Anomalies? J Perinatol. 2016;36(5):376-380. doi:10.1038/jp.2015.203.

19. Palmeri L, Gradwohl G, Nitzan M, et al. Photoplethysmographic Waveform Characteristics of Newborns with Coarctation of the Aorta. J Perinatol. 2017;37(1):77-80. doi:10.1038/jp.2016.162.

20. Amirtharaj CR, Palmeri LC, Gradwohl G, et al. Photoplethysmographic Assessment of Pulse Transit Time Correlates with Echocardiographic Measurement of Stroke Volume in Preterm Infants with Patent Ductus Arteriosus. J Perinatol. 2018;38(9):1220-1226. doi:10.1038/s41372-018-0165-0.

21. de-Wahl Granelli A, Ostman-Smith I. Noninvasive Peripheral Perfusion Index as a Possible Tool for Screening for Critical Left Heart Obstruction. Acta Paediatr. 2007;96(10):1455-1459. doi:10.1111/j.1651-2227.2007.00439.x.

22. Schena F, Picciolli I, Agosti M, et al. Perfusion Index and Pulse Oximetry Screening for Congenital Heart Defects. J Pediatr. 2017;183:74-79. doi:10.1016/j.jpeds.2016.12.076.

23. Searle J, Thakkar DD, Banerjee J. Does Pulsatility Index Add Value to Newborn Pulse Oximetry Screening for Critical Congenital Heart Disease? Arch Dis Child. 2018;(November):archdischild-2018-315891. doi: 10.1136/archdischild-2018-315891.

24. Kemper AR, Mahle WT, Martin GR, et al. Strategies for Implementing Screening for Critical Congenital Heart Disease. Pediatrics. 2011;128(5):e1259-67. doi:10.1542/peds.2011-1317.

25. Jegatheesan P, Nudelman M, Goel K, Song D, Govindaswami B. Perfusion Index in Healthy Newborns During Critical Congenital Heart Disease Screening at 24 hours: Retrospective Observational Study from the USA. BMJ Open. 2017;7(12):e017580. doi:10.1136/bmjopen-2017-017580.

26. Uygur O, Koroglu OA, Levent E, et al. The Value of Peripheral Perfusion Index Measurements for Early Detection of Critical Cardiac Defects. Pediatr Neonatal. 2018:1-6. doi:10.1016/j.pedneo.2018.04.003.

27. Chang R-KR, Gurvitz M, Rodriguez S. Missed Diagnosis of Critical Congenital Heart Disease. Arch Pediatr Adolesc Med. 2008;162(10):969. doi:10.1001/archpedi.162.10.969

28. Martin JA, Hamilton BE, Osterman MJK, Driscoll AK, Drake P. National Vital Statistics Reports Births: Final Data for 2016. Natl Vital Stat Reports. 2018;67(1):1-55. http://www.mendeley.com/research/deaths-final-data-for-2002-national-vital-statistics-reports/.

29. Diller CL, Kelleman MS, Kupke KG, Quary SC, Kochilas LK, Oster ME. A Modified Algorithm for Critical Congenital Heart Disease Screening Using Pulse Oximetry. Pediatrics. 2018;141(5).

30. Kochilas LK, Lohr JL, Bruhn E, et al. Implementation of Critical Congenital Heart Disease Screening in Minnesota. Pediatrics. 2013;132(3):e587-94. doi:10.1542/peds.2013-0803

31. Siefkes H, Kair L, Tancredi DJ, et al. Oxygen Saturation and Perfusion Index-Based Enhanced Critical Congenital Heart Disease Screening. Am J Perinatol. 2020;37(2):158-165. doi:10.1055/s-0039-1685445

32. Dash M, Liu H. Feature Selection for Classification. Intell Data Anal. 1997;1(1-4):131-156. doi:doi:10.1016/S1088-467X(97)00008-5.

33. Chawla N, Bowyer K, Hall L, Kegelmeryer W. SMOTE: Synthetic Minority Over sampling Technique. J Artif Intell Res. 2002;16:321-357.

34. Peterson C, Ailes E, Riehle-Colarusso T, et al. Late detection of critical congenital heart disease among us infants stimation of the potential impact of proposed universal screening using pulse oximetry. JAMA Pediatr. 2014;168(4):361-370. doi:10.1001/jamapediatrics.2013.4779

35. Sorensen MW, Sadiq I, Clifford GD, Maher KO, Oster ME. Using Pulse Oximetry Waveforms to Detect Coarctation of the Aorta. Biomed Eng Online. 2020:1-12. doi:10.1186/s12938-020-00775-2

36. Mills DL. Network Time Protocol Version 4 Reference and Implementation Guide. 2006;(June):1-90.

37. Sebelius K. Secretary of Health & Human Services Letter to the Secretary’s Advisory Committee on Heritable Disorders in Newborns and Children (SACHDNC). https://www.hrsa.gov/sites/default/files/hrsa/advisory-committees/heritable-disorders/reports-recommendations/response-congenital-cyanotic.pdf.

38. Oyonarte M, Dickinson D, Medici D, Hamilton D. Indirect Arterial Pulse Tracings in Children with Coarctation of the Aorta Before and After Operation. Thorax. 1980;35:128-132.

39. Oster ME, Aucott SW, Glidewell J, et al. Lessons Learned From Newborn Screening for Critical Congenital Heart Defects. Pediatrics. 2016;137(5):e20154573-e20154573. doi:10.1542/peds.2015-4573

40. K. Doshi, G. B. Rehm, P. Vadlaputi, Z. Lai, S. Lakshminrusimha, C.N. Chuah, and H. M. Siefkes, “A novel system to collect dual pulse oximetry data for critical congenital heart disease screening research,” J. Clin. Transl. Sci., pp. 1-7.

41. G. R. Martin, A. K. Ewer, A. Gaviglio, L. A. Hom, A. Saarinen, M. Sontag, K. M. Burns, A. R. Kemper, and M. E. Oster, “Updated strategies for pulse oximetry screening for critical congenital heart disease,” Pediatrics, vol. 146, no. 1, 2020.

42. L. Wei, S. Ventura, M. Lowery, M. A. Ryan, S. Mathieson, G. B. Boylan, and C. Mooney, “Random forest-based algorithm for sleep spindle detection in infant eeg,” in 2020 42nd Annual International Conference of the IEEE Engineering in Medicine & Biology Society (EMBC). IEEE, 2020, pp. 58-61.

43. B. I. Ferleger, K. S. Sonnet, T. H. Morriss, A. L. Ko, H. J. Chizeck, and J. A. Herron, “A tablet-and mobile-based application for remote diagnosis and analysis of movement disorder symptoms,” in 2020 42nd Annual International Conference of the IEEE Engineering in Medicine & Biology Society (EMBC). IEEE, 2020, pp. 5588-5591.

44. M. N. Plana, J. Zamora, G. Suresh, L. Fernandez-Pineda, S. Thangaratinam, and A. K. Ewer, “Pulse oximetry screening for critical congenital heart defects,” Cochrane Database Syst. Rev., no. 3, 2018.

45. S. B. Cantor and M. W. Kattan, “Determining the area under the roc curve for a binary diagnostic test,” Med. Decis. Mak., vol. 20, no. 4, pp. 468-470, 2000.

Other aspects are set forth within the following claims.

What is claimed is:

1. A computer-implemented method comprising:
obtaining, via a first oximeter probe secured to an upper extremity of a patient and/or a second oximeter probe secured to a lower extremity of the patient, a plurality of physiological measurements from the patient;
applying a predictive model to the plurality of physiological measurements from the patient to generate a classification corresponding to a vascular condition, the predictive model having been trained, using a machine learning system, by:
acquiring, using one or more pulse oximeters, physiological readings from subjects in a study cohort;
extracting a set of features from the physiological readings to generate a training dataset based on the physiological readings from the subjects in the study cohort; and
applying machine learning techniques to the training dataset to train the predictive model such that the predictive model is configured to accept the plurality of physiological measurements and generate a classification corresponding to the vascular condition, wherein applying the machine learning techniques comprises performing automated feature selection to identify a subset of the set of features and refitting the predictive model based on the subset of features, wherein the subset of features corresponds to the plurality of physiological measurements; and outputting or storing the classification in association with the patient.

2. The method of claim 1, wherein the vascular condition is a congenital heart disease.

3. The method of either claim 1 or claim 2, wherein the patient and the subjects in the cohort are newborns and/or infants.

4. The method of any of claims 1-3, wherein the upper extremity is a hand or wrist.

5. The method of any of claims 1-4, wherein the lower extremity is a foot or ankle.

6. The method of any of claims 1-5, wherein the upper extremity is a preductal site.

7. The method of any of claims 1-6, wherein the lower extremity is postductal site.

8. The method of any of claims 1-7, wherein the patient and the subjects in the cohort are adults.

9. The method of any of claims 1-8, wherein the machine learning techniques comprises a random forest classifier.

10. The method of any of claims 1-9, wherein the machine learning techniques comprises logistic regression.

11. The method of any of claims 1 - 10, wherein the machine learning techniques comprises an ensemble of a random forest classifier and logistic regression.

12. The method of any of claims 1 - 11, wherein the machine learning techniques comprises a random forest classifier, logistic regression, a Naive Bayes Classifier, a K-Nearest Neighbours algorithm, a Decision Tree, a Support Vector Machine algorithm, and/or a Gradient Boosting Classifier.

13. The method of any of claims 1 - 12, further comprising securing the first oximeter probe to the right hand of the patient, and securing the second oximeter probe to either foot of the patient.

14. The method of any of any of claims 1 - 13, wherein the subset of features comprises oxygen saturation (SpO2).

15. The method of any of any of claims 1 - 14, wherein the subset of features comprises heart rate (HR).

16. The method of any of any of claims 1 - 15, wherein the subset of features comprises perfusion amplitude index (PAI).

17. The method of any of claims 1 - 16, wherein the subset of features comprises oxygen saturation (SpO2) and perfusion amplitude index (PAI).

18. The method of any of claims 1 - 17, wherein the subset of features comprises oxygen saturation (SpO2) and heart rate.

19. The method of any of claims 1 - 18, wherein the subset of features comprises perfusion amplitude index (PAI) and heart rate.

20. The method of any of claims 1 - 19, wherein the subset of features comprises oxygen saturation (SpO2), heart rate (HR), and perfusion amplitude index (PAI).

21. The method of any of claims 1 - 20, wherein the subset of features comprises maximum HR, HR variance, median SpO2, mean SpO2, and mean PAI.

22. The method of any of claims 1 - 21, wherein the subset of features comprises minimum HR, maximum HR, HR variance, median SpO2, mean SpO2, mean PAI (or PIx), and minimum PAI.

23. The method of any of claims 1 - 22, wherein the subset of features comprises median HR, mean HR, maximum HR, HR variance, minimum SpO2, maximum SpO2, median SpO2, mean SpO2, mean PAI (or PIx), median PAI (or PIx), and maximum PAI (or PIx).

24. The method of any of claims 1 - 23, wherein the performing automated feature selection comprises performing Recursive Feature Elimination (RFE).

25. The method of any of claims 1 - 24, wherein the performing automated feature selection comprises performing Recursive Feature Elimination (RFE) with sensitivity selected as a score to be optimized.

26. The method of any of claims 1 - 25, wherein the physiological readings from the subjects are acquired over a predetermined time period.

27. The method of claim 26, wherein the time period is at least one minute.

28. The method of claim any of claims 1 - 27, further comprising displaying, on a display screen, physiological readings sensed via the first and second oximeter probes in real time or near real time.

29. A method comprising using a machine learning system to train a machine learning predictive model by:
acquiring, using one or more pulse oximeters, physiological readings from subjects in a study cohort for a time period;
extracting a set of features from the physiological readings to generate a training dataset based on the physiological readings from the subjects in the study cohort; and
applying machine learning techniques to the training dataset to train the predictive model such that the predictive model is configured to accept data based on a plurality of physiological measurements from patients and generate classifications corresponding to a vascular condition, wherein the training dataset comprises a set of features, and wherein applying the machine learning techniques comprises performing automated feature selection to identify a subset of the set of features and refitting the predictive model based on the subset of features.

30. The method of claim 29, wherein the vascular condition is a congenital heart disease.

31. The method of either claim 29 or claim 30, wherein the subjects in the cohort are newborns.

32. The method of any of claims 29 - 31, wherein a first oximeter probe is secured to the right hand of each of the subjects, and a second oximeter probe is secured to either foot of each of the subjects.

33. The method of any of claims 29 - 32, wherein the subset of features comprises oxygen saturation (SpO2), heart rate (HR) and perfusion amplitude index (PAI).

34. The method of any of claims 29 - 33, wherein the performing automated feature selection comprises performing Recursive Feature Elimination (RFE).

35. The method of any of claims 29 - 34, wherein the physiological readings from the subjects are acquired over a time period of at least three minutes.

36. The method of any of claims 29 - 35, further comprising:
acquiring, using one or more pulse oximeters, a plurality of physiological readings from a patient; and
applying the predictive model to a plurality of physiological measurements based on the physiological readings from the patient to generate a classification corresponding to the vascular condition.

37. A method comprising:
acquiring, by one or more processors, using one or more pulse oximeters, oxygen saturation (SpO2) and perfusion index (PIx) data from subjects in a study cohort to generate a training dataset;
applying machine learning techniques to the training dataset to train a predictive model such that the predictive model is configured to accept SpO2 and PIx data and generate a classification corresponding to a vascular condition;
acquiring, by the one or more processors, using one or more pulse oximeters, SpO2 and PIx data from a patient;
applying, by the one or more processors, the predictive model to the SpO2 and PIx data from the patient to generate the classification corresponding to the vascular condition.

38. The method of claim 37, wherein the vascular condition is a congenital heart defect, and wherein the subjects and the patient are newborns.

39. The method of either claim 37 or claim 38, wherein the classification corresponds to at least one of a presence or a severity of the vascular condition.

40. The method of any of claims 37 - 39, further comprising acquiring heart rate data from the patient.

41. A computer-implemented method of classifying congenital heart defects in fetuses, newborns, or infants, the method comprising:
acquiring, by one or more processors, using one or more pulse oximeters, oxygen saturation (SpO2) and perfusion index (PIx) data from a study cohort to generate a training dataset;
applying machine learning techniques to a training dataset based on the SpO2 and Pix data to train a predictive model such that the predictive model is configured to accept SpO2 and PIx data and generate a classification corresponding to at least one of a presence or a severity of a congenital heart defect;
acquiring, by the one or more processors, using one or more pulse oximeters, SpO2 and PIx data from a subject;
applying, by the one or more processors, the predictive model to the SpO2 and PIx data from the subject to generate the classification as to whether the congenital heart defect is detected in the subject.

42. The method of claim 41, wherein the predictive model is further configured to accept radiofemoral delay for use in generating the classification.

43. The method of claim 42, wherein the radiofemoral delay is based on simultaneous hand and foot measurements.

44. The method of any of claims 41 - 43, wherein the predictive model is further configured to accept photoplethysmography (PPG) waveform data for use in generating the classification.

45. The method of claim 44, wherein the PPG waveform data comprises PPG waveform slope.

46. The method of claim 44, wherein the PPG waveform data comprises one or more PPG waveform images.

47. The method of any of claims 41 - 46, wherein the predictive model is further configured to accept heart rate data for use in generating the classification.

48. The method of claim 47, wherein the heart rate data comprises heart rate measurements.

49. The method of claim 47, wherein the heart rate data comprises heart rate variability data.

50. A system comprising a computing device and one or more pulse oximeters, the computing device comprising a controller configured to:
acquire, from the one or more pulse oximeters, oxygen saturation (SpO2) and perfusion index (PIx) measurements from a patient; and
apply a predictive model to a set of patient data comprising the SpO2 and PIx measurements from the patient to generate a classification as to whether a heart defect is detected in the patient.

51. The system of claim 50, wherein the controller is further configured to train the predictive model by:
acquiring, from one or more pulse oximeters, SpO2 and PIx data from a study cohort to generate a training dataset; and
using the training dataset to train the predictive model such that the predictive model is configured to accept SpO2 and PIx data and generate the classification as to whether the heart defect is detected.

52. The system of either claim 50 or claim 51, wherein the controller is further configured to obtain radiofemoral delay, and wherein the set of patient data further comprises the radiofemoral delay.

53. The system of claim 52, wherein the radiofemoral delay is based on simultaneous hand and foot measurements.

54. The system of any of claims 50 - 53, wherein the controller is further configured to obtain heart rate measurements, and wherein the set of patient data further comprises the heart rate measurements.

55. The system of any of claims 50 - 54, wherein the controller is further configured to obtain heart rate variability data, and wherein the set of patient data further comprises the heart rate variability data.

56. The system of any of claims 50 - 55, wherein the controller is further configured to obtain photoplethysmography (PPG) waveform data, and wherein the set of patient data further comprises the PPG waveform data.

57. The system of claim 56, wherein the PPG waveform data comprises a PPG waveform slope, and wherein the set of patient data further comprises the PPG waveform slope.

58. The system of claim 56, wherein the PPG waveform data comprises a PPG waveform image, and wherein the set of patient data further comprises the PPG waveform image.

59. A computer-implemented method comprising:
acquiring, by a controller of a computing device using one or more pulse oximeters, oxygen saturation (SpO2) and perfusion index (PIx) measurements from a patient; and
applying, by the controller, a predictive model to a set of patient data comprising the SpO2 and PIx measurements from the patient to generate a classification as to whether a heart defect is detected in the patient.

60. The method of claim 59, wherein the predictive model is trained by:
acquiring, by the controller, using one or more pulse oximeters, SpO2 and PIx data from a study cohort to generate a training dataset; and
using, by the controller, the training dataset to train the predictive model such that the predictive model is configured to accept SpO2 and PIx data and generate the classification as to whether the heart defect is detected.
